(19) United States
(12) Patent Application Publication
(10) Pub. No.: US 2026/0232777 A1
(43) Pub. Date: Aug. 13, 2026

(54) PVAX14 NUCLEIC ACID FORMULATED WITH NANO-VEHICLE IN COMBINATION WITH ALL-TRANS RETINOIC ACID (ATRA) FOR ACTIVATING IMMUNE PATHWAYS

(71) Applicants: VIVADJU, Paris (FR); INSTITUT NATIONAL DE LA SANTE ET DE LA RECHERCHE MEDICALE, Paris (FR); CENTRE NATIONAL DE LA RECHERCHE SCIENTIFIQUE, Paris (FR); UNIVERSITÉ PARIS CITÉ, Paris (FR); UNIVERSITÉ DE NANTES, Nantes (FR); UNIVERSITÉ D'ANGERS, Angers (FR); ASSISTANCE PUBLIQUE-HOPITAUX DE PARIS, Paris (FR)

(72) Inventors: Rose Ann PADUA, Paris (FR); Christine CHOMIENNE, Bois Le Roi (FR); Marie PLA, Paris (FR); Patricia KRIEF, Gonesse (FR); Bruno PITARD, Nantes (FR)

(21) Appl. No.: 19/469,794

(22) PCT Filed: Mar. 29, 2024
(86) PCT No.: PCT/EP2024/058784

§ 371 Date: Sep. 26, 2025

(30) Foreign Application Priority Data
Mar. 30, 2023 (EP) 23305447.7

(51) Int. Cl. A61K 39/00; A61K 9/51; A61K 31/203; A61K 31/706; A61K 31/7105; A61K 33/36; A61P 35/00; C12N 15/113; C12N 15/85; C12Q 1/6809; C12Q 1/6886

(52) U.S. Cl. CPC A61K 39/0011; A61K 9/5153; A61K 31/203; A61K 31/706; A61K 31/7105; A61K 33/36; A61P 35/00; C12N 15/113; C12N 15/85; C12Q 1/6809; C12Q 1/6886; A61K 2039/53; A61K 2039/55555; C12N 2310/11; C12Q 2600/106; C12Q 2600/158

(57) ABSTRACT

The present invention relates to a pharmaceutical combination comprising (i) at least one nucleic acid comprising an antisense and flipped sequence of a fragment of a sequence coding for a kanamycin resistance protein and (ii) at least one non-immunosuppressive inducer of tumor cell apoptosis, wherein said nucleic acid is formulated with a nano-vehicle. Said nucleic acid is preferably pVAX14 plasmid, said non-immunosuppressive inducer of tumor cell apoptosis is preferably ATRA, an arsenic-related compound or azacytidine. The present invention also relates to the pharmaceutical combination as defined above for use in the treatment of cancer or infectious diseases, by activating the innate immune pathway and immunological cell death pathway. Pathway analyses of gene expression profiles revealed a DNA activated gene list regulated in 16 immune pathways. This gene list provides biomarkers of response to DNA treatment.

### FIELD OF THE INVENTION

The present invention relates to the treatment of cancer.

### TECHNICAL BACKGROUND

The development of malignancy is a two-edged sword with an expansion of abnormal cells that acquire properties of stem cells and the increasing immunosuppression as disease progresses. Acquisitions of stemness and immuno-inhibition are directly associated with specific changes of gene expression programs associated with epigenetic regulation, which is now considered a bridge between genotype and phenotype integrating both intrinsic (gene mutation) and environmental (crosstalk between cancer cell and biological environment) signals. The microenvironment includes the immune system and how the tumor cells interact with the immune system can be exploited.

The Inventors have shown in mouse models that DNA plasmids containing either specific or nonspecific sequences enhance survival when combined with a differentiating/apoptotic and immunomodulators, such as a vitamin A derivative, all-trans retinoic acid (ATRA). A specific DNA plasmid containing sequences around the fusion gene of PML-RARa has previously been shown in acute promyelocytic leukemia (APL) mice to enhance survival when combined with ATRA, with a reduction of minimal residual disease (MRD) (Furugaki et al, 2010, Blood 15:653-656; Padua et al, 2003; Pokorna et al, 2013). The Inventors developed a DNA plasmid construct pVAX14 in combination with the immunomodulatory all-trans retinoic acid (ATRA), to activate and enhance the immune system in all patients with cancers, the cancer cell itself having provided the antigen(s) to mount the tumor specific responses. The efficacy of this nonspecific vaccine pVAX14 was found to be similar to that of the specific vaccine in the APL model and significantly different from ATRA alone, p<0.0014) (Le Pogam et al, 2015, Oncotarget 6:32494-32508).

Enhanced survival correlated with enhanced immune responses and the control or targeting of disease in the APL mouse model (Furugaki et al, 2010; Padua et al, 2003; Pokorna et al, 2013; Robin et al, 2006, Blood 108:1972-1974; Le Pogam et al, 2015; Patel et al, 2015, Blood Cancer Journal Dec 11;5(12):e374. doi: 10.1038/bcj.2015.102) and similar results were obtained in a mouse model of high risk myelodysplasia (HR-MDS) (Le Pogam et al, 2015; Omidvar et al, 2007); this correlated with increased immune responses and disease control.

There is still a need of improved treatments of cancer and also of methods for monitoring the response to such treatments in patients.

### DESCRIPTION OF THE INVENTION

The Inventors have unexpectedly found that the combined use of (i) a nucleic acid comprising an antisense and flipped sequence of a fragment of a sequence coding for the kanamycin resistance protein, such as pVAX14, formulated with a nano-vehicle, and (ii) at least one non-immunosuppressive inducer of tumor cell apoptosis, such as ATRA, allows upregulating genes of the innate immune pathway, in particular HMGB1, RIG-1 and MYD88 genes. In addition, the inflammasome pathway is also activated with increased apoptosis. For example, co-culturing effectors isolated from pVAX14+ATRA treated AML (Acute Myeloid Leukaemia) mice with bone marrow target cells from AML mice results in an increased mRNA expression of Nlrp3 and detection of Caspase 1 protein. The nucleic acid delivered by a nano-vehicle, in particular a nanotaxi®, increases the expression of adaptors DHX9 and DHX36 to activate MyD88 (data not shown) and intracytoplasmic DNA sensor (DAI) to activate the inflammasome pathway (FIG. 1A).

Said nucleic acid (i) can advantageously be used in combination with ATRA and, optionally, with a second non-immunosuppressive inducer of tumor cell apoptosis, such as arsenic trioxide or azacytidine.

Besides, uptake of the nucleic acid comprising the antisense and flipped sequence is advantageously increased when delivered in a nano-vehicle, such as in nanotaxi®. For example, the combination of the DNA plasmid pVAX14 delivered by nanotaxi® and ATRA was used in a transplantable model of AML 3 and was shown to decrease the PML-RARA transcript compared to naked DNA (FIG. 1B).

The Inventors also unexpectedly found a panel of 29 biomarkers allowing detecting activation of an immune response. At least 5 biomarkers of said panel may advantageously be used to monitor the response to a treatment based on the combined use of the nucleic acid as defined above and the non-immunosuppressive inducer of tumor cell apoptosis as defined above.

A first object of the inventions is thus a pharmaceutical combination comprising:
(i) at least one nucleic acid comprising an antisense and flipped sequence of a fragment of the sequence coding for a kanamycin resistance protein, wherein said nucleic acid is capable of eliciting an immune response in a mammal and wherein said nucleic acid is formulated with a nano-vehicle, and
(ii) at least one non-immunosuppressive inducer of tumor cell apoptosis.

Said nucleic acid preferably comprises a sequence selected from the group consisting of:
a) sequence SEQ ID NO: 2,
b) a sequence at least 80% identical to SEQ ID NO: 2,
c) a fragment of at least 50 consecutive nucleotides of SEQ ID NO: 2, and
d) sequence SEQ ID NO: 3.

Said antisense and flipped sequence preferably encodes at least one immunogenic peptide selected from the group consisting of sequence SEQ ID NO: 4 and SEQ ID NO: 5.

Said nucleic acid preferably encodes an immunogenic peptide of sequence SEQ ID NO: 6.

Said non-immunosuppressive inducer of tumor cell apoptosis is preferably ATRA, arsenic, arsenic trioxide (ATO) or azacytidine.

In the pharmaceutical combination as defined above, said nucleic acid (i) and said non-immunosuppressive inducer of tumor cell (ii) are preferably provided in the form of separate pharmaceutical compositions.

In the pharmaceutical combination as defined above, each of said nucleic acid (i) and said non-immunosuppressive inducer of tumor cell apoptosis (ii) may be provided in the form of one or more doses of a pharmaceutical composition.

Another object of the invention is the pharmaceutical combination as defined above, for use as a medicament, preferably in the treatment of cancer, such as a solid tumor, preferably breast cancer, or a blood cancer, or in the treatment of infectious diseases.

Said nucleic acid (i) and said non-immunosuppressive inducer of tumor cell apoptosis (ii) may be administered simultaneously or sequentially.

Said nucleic acid (i) and/or said non-immunosuppressive inducer of tumor cell apoptosis (ii) may be administered by intradermal, intra-epidermal, subcutaneous, intramuscular, intravenous, oral route or intratumoral route.

The present invention also relates to the pharmaceutical combination for use as defined above, wherein:
said nucleic acid (i) is administered at a dosage of from 500 μg to 2 mg, and/or
said non-immunosuppressive inducer of tumor cell apoptosis (ii) is ATRA and is administered at a dosage of 45 mg/m².

The present invention also relates to the pharmaceutical combination for use as defined above, wherein:
said nucleic acid (i) is administered once or at least twice, preferably with an interval of two weeks between two administrations, and/or
said non-immunosuppressive inducer of tumor cell apoptosis (ii) is preferably ATRA and is administered once a day for 10 days, each time said nucleic acid (i) is administered.

The present invention also relates to the pharmaceutical combination for use as defined above, wherein a second non-immunosuppressive inducer of tumor cell apoptosis is used. Said second non-immunosuppressive inducer of tumor cell apoptosis is preferably arsenic trioxide or azacytidine.

Another object of the invention is a method for assessing activation of an immune response, wherein said method comprises measuring the quantity of at least 5 biomarkers selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2 in a biological sample.

Another object of the invention is a method for monitoring response to a treatment based on the pharmaceutical combination as defined above in a subject, wherein said method comprises:
a) measuring the quantity of at least 5 biomarkers selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2 in a biological sample from said subject,
b) comparing the results obtained at step a) with a corresponding control value and/or to a corresponding value from said subject measured before beginning the treatment or measured during the course of treatment,
c) deducing if said subject responds favorably to the treatment, and
d) optionally, repeating steps a) to c).

Another object of the invention is a kit suitable for assessing activation of an immune response, wherein said kit comprises means for detecting at least 5 biomarkers selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2.

Another object of the invention is an in vitro use of the kit as defined above for assessing activation of an immune response.

### Nucleic Acid Comprising an Antisense and Flipped Sequence of a Fragment of the Sequence Coding for the Kanamycin Resistance Protein

The pharmaceutical combination according to the invention comprises at least one nucleic acid comprising an antisense and flipped sequence of a fragment of a sequence coding for the kanamycin resistance protein.

By “antisense and flipped sequence of sequence X”, it is herein meant the reverse complement sequence of sequence X.

This means that said antisense and flipped sequence of a fragment of a sequence coding for the kanamycin resistance protein does not encode a kanamycin resistance protein, nor a fragment thereof.

However, using an antisense and flipped sequence allowed creating novel coding sequences, in particular encoding new immunogenic peptides.

Consequently, the nucleic acid as defined above is capable of eliciting an immune response in a mammal, such as in a mouse, rat or human.

Methods for measuring an immune response are well known in the art. For example, the methods disclosed in Padua et al. (2003, Nature Medicine 9:1413-1417) may be used.

Immune response may also be measured by the method disclosed below, which uses an immune gene signature generated by the pathway analysis of genes regulated measured by RNA-sequence arrays.

By “nucleic acid”, it is herein meant to the phosphate ester polymeric form of ribonucleosides (“RNA”) or deoxyribonucleosides (“DNA”), or any phosphoester analogs thereof, such as phosphorothioates and thioesters, in particular in either single stranded form or a double-stranded helix.

The nucleic acid as defined above is preferably a double-stranded DNA.

The nucleic acid as defined above may be a linear or circular DNA.

The nucleic acid as defined above is preferably an isolated nucleic acid.

By the term “isolated”, it is herein particularly meant a compound, which is isolated from a human or animal body, from a cell and/or from a library of compounds.

As used herein, the term “kanamycin resistance protein” refers to an enzyme capable of conferring resistance to kanamycin.

Preferably, the sequence coding for the kanamycin resistance protein consists of nucleotides 1226 to 2020 of sequence SEQ ID NO: 7.

The nucleic acid as defined above may further comprise a reverse complement sequence of a fragment of pVAX1 plasmid, in particular a reverse complement sequence of a fragment of pVAX1 plasmid, which is adjacent to the sequence coding for the kanamycin resistance protein.

The sequence of the pVAX1 plasmid is shown as SEQ ID NO: 7.

Said fragment of the sequence coding for the kanamycin resistance protein may be of any length, e.g. of at least, at most or about 50, 100, 150, 200, 250, 300, 350, 400, 450, 473 or 500 consecutive nucleotides.

Said fragment for example consists of from 50 to 500 consecutive nucleotides, preferably from 100 to 500 consecutive nucleotides, for example from 100 to 473 consecutive nucleotides.

The antisense and flipped sequence as defined above preferably comprises a sequence selected from the group consisting of:
a) a reverse complement sequence of a fragment of SEQ ID NO: 7, wherein said fragment comprises at least 50 consecutive nucleotides of the nucleotides located from position 1226 to position 2020 of SEQ ID NO: 7;
b) sequence SEQ ID NO: 1;
c) a sequence at least 80%, preferably at least 85%, at least 90%, at least 95%, at least 96%, at least 97%, at least 98% or at least 99% identical to SEQ ID NO:1;
d) a fragment of at least 50, preferably at least 100, at least 150, at least 200, at least 250, at least 300, at least 350, at least 400 or at least 450, consecutive nucleotides of SEQ ID NO: 1; and
e) a derivative of (a) or (b) capable of eliciting an immune response in a mammal.

The antisense and flipped sequence as defined above may for example comprise or consist of the reverse complement sequence of a fragment of SEQ ID NO: 7, wherein said fragment of SEQ ID NO: 7 comprises at least, at most or about 50, 100, 150, 200, 250, 300, 350, 400, 450, 473 or 500 consecutive nucleotides of the nucleotides located from position 1226 to position 2020 of SEQ ID NO: 7.

The fragment of SEQ ID NO: 7 may for example comprise or consist of 50 to 500, 100 to 473, 100 to 500, 200 to 500, 300 to 500, 400 to 500 or 450 to 500 consecutive nucleotides of the nucleotides located from position 1226 to position 2020 of SEQ ID NO: 7.

The antisense and flipped sequence as defined above preferably encodes at least one immunogenic peptide selected from the group consisting of sequence SEQ ID NO: 4 and SEQ ID NO: 5.

The immunogenic peptide of sequence SEQ ID NO: 4 corresponds to the peptide encoded by ORF1 starting at position 813 in sequence SEQ ID NO: 3,

The immunogenic peptide of sequence SEQ ID NO: 5 corresponds to the peptide encoded by ORF2 starting at position 818 in sequence SEQ ID NO: 3.

The nucleic acid as defined above preferably comprises a sequence selected from the group consisting of:
a) sequence SEQ ID NO: 2;
b) a sequence at least 80%, preferably at least 85%, at least 90%, at least 95%, at least 96%, at least 97%, at least 98% or at least 99% identical to SEQ ID NO: 2;
c) a fragment of at least 50, preferably at least 100, at least 150, at least 200, at least 250, at least 300, at least 350, at least 400 or at least 450, consecutive nucleotides of SEQ ID NO: 2; and
d) a derivative of SEQ ID NO: 2 capable of eliciting an immune response in a mammal.

The nucleic acid as defined above preferably encodes an immunogenic peptide of sequence SEQ ID NO: 6.

The immunogenic peptide of sequence SEQ ID NO: 6 corresponds to the peptide encoded by ORF5 starting at position 1432 of sequence SEQ ID NO: 3.

The nucleic acid as defined above may further comprise a sequence that encodes a tumor antigen.

In another embodiment, the nucleic acid as defined above does not comprise any sequence that encodes a tumor antigen.

By the expression “nucleic acid having a sequence at least x % identical to sequence Y”, it is herein meant that the sequence of the nucleic acid is identical to sequence Y except that the sequence may include up to 100-x nucleotide alterations per each 100 nucleotides of the query sequence. In other words, to obtain a nucleic acid having a sequence at least x % identical to a query sequence, up to 100-x % of the nucleotides of the sequence may be inserted, deleted, or substituted with another nucleotide. “x” is preferably comprised between 80 and 100.

The term “derivative” includes homologues, mutants and naturally occurring variants such as allelic variants, splice variants or variants obtained through proteolytic processing. Derivatives consisting of an amino acid sequence at least x % identical to a reference sequence may comprise mutation(s), such as deletion(s), insertion(s) and/or substitution(s) compared to the reference sequence. In case of substitutions, the derivative consisting of an amino acid sequence at least x % identical to a reference sequence may correspond to a homologous sequence derived from another species than the reference sequence. The substitution may correspond to a conservative substitution as indicated in the table below.

| Conservative substitutions | Type of Amino Acid |
|---|---|
| Ala, Val, Leu, Ile, Met, | Amino acids with aliphatic |
| Pro, Phe, Trp | hydrophobic side chains |
| Ser, Tyr, Asn, Gln, Cys | Amino acids with uncharged but |
| | polar side chains |
| Asp, Glu | Amino acids with acidic side chains |
| Lys, Arg, His | Amino acids with basic side chains |
| Gly | Neutral side chain |

The nucleic acid as defined above may be provided in the form of a vector.

The nucleic acid as defined above is preferably a vector, for example a plasmid.

In said vector, the antisense and flipped sequence is preferably placed under the control of signals (e.g. a promoter, a terminator and/or an enhancer) allowing the transcription of the antisense and flipped sequence.

The vector as defined above is preferably a DNA vaccination vector, i.e. a vector specifically designed for the development of a DNA vaccine.

The DNA vaccination vector may correspond to or be derived from, e.g. the pVAX1 of sequence SEQ ID NO: 7 (Invitrogen, Carlsbad, California, USA) or the pCDNA₃ (Invitrogen, Carlsbad, California, USA) expression vector.

The vector as defined above may further comprise a resistance gene, for example conferring resistance to an antibiotic, such as ampicillin or kanamycin.

Alternatively, the vector as defined above does not comprise any gene conferring resistance to an antibiotic.

The vector as defined above preferably comprises sequence SEQ ID NO: 2.

The vector as defined above preferably allows the expression of at least one immunogenic peptide selected from the group consisting of sequence SEQ ID NO: 4, SEQ ID NO: 5 and SEQ ID NO: 6.

The vector as defined above preferably allows the expression of immunogenic peptide SEQ ID NO: 4, immunogenic peptide SEQ ID NO: 5 and, optionally, immunogenic peptide SEQ ID NO: 6.

A preferred vector as defined above comprises or consists of sequence SEQ ID NO: 3.

In one preferred embodiment, the nucleic acid as defined above comprises sequence SEQ ID NO: 3, more preferably is the pVAX14 plasmid of sequence SEQ ID NO: 3.

### Nano-Vehicle

The pharmaceutical combination according to the invention comprises at least one nucleic acid as defined above in the section “Nucleic acid comprising an antisense and flipped sequence of a fragment of the sequence coding for the kanamycin resistance protein”, which is formulated with a nano-vehicle.

By the expression “formulated with a nano-vehicle” or “delivered by a nano-vehicle”, it is herein meant that the nucleic acid is vectorized using a nano-vehicle. In particular, the nano-vehicle self-assembles with at least one nucleic acid, thereby forming a supramolecular structure.

By “nano-vehicle”, it is herein meant an amphiphilic block copolymer with a tetra arm centered around an ethylidiamine structure.

By “block copolymer”, it is herein meant a polymer comprising at least two sets, or blocks, of polymerized monomeric units. A “block” refers to a motif, obtained by polymerization of a monomer, and which may be repeated within the polymer.

By “amphiphilic block copolymer”, it is herein meant a block copolymer comprising at least one hydrophilic block and at least one hydrophobic block.

Such amphiphilic block copolymers are for example disclosed in documents EP1471944 or EP2819683 and are also referred to as Nanotaxi®.

The nano-vehicle as defined above, also referred to as Nanotaxi®, is for example a tetra-functional amphiphilic block copolymer of formula (I):

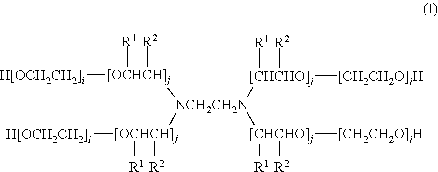

or an organic or mineral salt thereof,
in which i and j represent, independently of one another, an integer of between 1 and 500, and
for each R1, R2 pair, one is hydrogen and the other is a methyl group.

In the formula (I) above, i has preferably a value such that said molecule comprises at least 30% by weight of ethylene oxide units and, preferably, no more than 85% by weight of ethylene oxide units. For example, the compound of formula (I) may comprise between 35% and 50%, preferably around 40% by weight of ethylene oxide units.

In the formula (I) above, i has preferably a value from 10 to 60 and j has preferably a value from 13 to 20. In a preferred embodiment, i is 13 and j is 14.

The mineral salt is preferably an alkali metal salt or an alkaline-earth metal salt. The mineral salt may for example be selected from the group consisting of sodium chloride, potassium chloride, lithium chloride, sodium thiocyanate, calcium chloride (CaCl2) and magnesium chloride (MgCl2).

The mineral salt may be in an isotonic, hypotonic or hypertonic amount.

The nano-vehicle as defined above may be used in a cationic form.

Alternatively, the nano-vehicle as defined above may be a glycosylated tetrafunctional non-ionic block copolymer, wherein said glycosyl moiety is preferably a mannose moiety.

A preferred nano-vehicle is a nano-vehicle as defined above comprising 13 units of ethylene oxide and 14 units of propylene oxide centered around an ethylenediamine. Such a preferred nano-vehicle has the following formula (II)

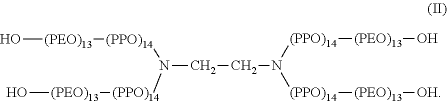

In the above formula (II), “PEO” means polyethylene oxide and “PPO” means polypropylene oxide.

Formulation of said nucleic acid in a nano-vehicle as defined above may be obtained by equivolumetric mixing of an aqueous solution of said nucleic acid, in particular at the desired concentration, with a solution comprising the nano-vehicle.

The pH of the solution comprising the nano-vehicle is preferably comprised from 6.5 to 8, more preferably from 7 to 7.8, for example 7.4.

The solution comprising the nano-vehicle preferably comprises a buffer, such as Tyrode's medium.

Tyrode's medium comprises 3 mM CaCl₂, 2 mM MgCl₂, 6 mM KCl, 140 mM NaCl, 10 mM glucose and 10 mM Hepes, pH 7.4.

Since the mixing of both solutions is equivolumetric, it is preferred that the aqueous solution comprising said nucleic acid is at 2× concentration and that the solution comprising the nano-vehicle and the buffer is also at 2× concentration, so as to get a final solution at 1× concentration.

### Non-Immunosuppressive Inducer of Tumor Cell Apoptosis

The pharmaceutical combination according to the invention further comprises at least one non-immunosuppressive inducer of tumor cell apoptosis, preferably one or two non-immunosuppressive inducers of tumor cell apoptosis.

Non-immunosuppressive inducers of tumor cell apoptosis are well-known in the art.

The non-immunosuppressive inducer of tumor cell apoptosis may for example be a retinoid compound, an arsenic-related compound, CD437, a compound activating CD44 (such as an antibody or hyaluronic acid), an hematopoietic growth and differentiation factor, 5-azacytidine, another demethylating agent, farnesyl transferase inhibitors (FTI), histone deacetylate inhibitors (HDACi), a small molecule such as Imatinib, a BH3 mimetic inhibitor, such as ABT 737, ABT-199, or a RAC1 inhibitor.

5-azacytidine is also referred to as azacytidine in the following.

By “retinoid compound”, it is herein meant a vitamin A derivative.

The retinoid compound may be selected from the group consisting of retinoic acid (RA), all-trans retinoic acid (ATRA), 9-cis RA, 4-HPPR, 13-cis RA and a synthetic analog of retinoic acid, such as AM 580.

Preferably, the retinoid compound is ATRA or AM 580.

ATRA is also referred to as tretinoin or all-trans retinoic acid.

ATRA is a derivative of vitamin A and is a ligand for the retinoic acid receptor (RAR).

ATRA is referred under CAS number 302-79-4.

AM 580 is an analog of retinoic acid.

AM 580 is referred under CAS number 102121-60-8.

By “arsenic-related compound”, it is herein meant any compound that, just as arsenic, is a phosphatase inhibitor or is capable of creating covalent bonds by dithiol group binding. The arsenic-related compound may for example be arsenic or arsenic trioxide (As₂O₃).

In a preferred embodiment, the non-immunosuppressive inducer of tumor cell apoptosis is arsenic, arsenic trioxide (ATO), all-trans retinoic acid (ATRA), azacytidine or AM 580.

In one embodiment, two non-immunosuppressive inducers of tumor cell apoptosis may be used in the pharmaceutical combination, such as for example ATRA and ATO or ATRA and azacytidine.

The non-immunosuppressive inducer of tumor cell apoptosis preferably has adjuvant activity towards the biological response elicited by the nucleic acid comprising the antisense and flipped sequence as defined above.

By “adjuvant activity”, it is herein meant an effect achieved by the combination of two components that is greater than the effect of either of the two components alone.

The non-immunosuppressive inducer of tumor cell apoptosis in the pharmaceutical combination is preferably ATRA, ATO or azacytidine.

A preferred non-immunosuppressive inducer of tumor cell apoptosis in the pharmaceutical combination is ATRA, optionally used in combination with a second non-immunosuppressive inducer of tumor cell apoptosis, such as ATO or azacytidine.

### Pharmaceutical Combination

The present invention particularly relates to a pharmaceutical combination comprising:
(i) at least one nucleic acid comprising an antisense and flipped sequence of a fragment of the sequence coding for a kanamycin resistance protein, as defined above, wherein said nucleic acid is formulated with a nano-vehicle, and
(ii) at least one non-immunosuppressive inducer of tumor cell apoptosis, as defined above, for example one or two non-immunosuppressive inducer(s) of tumor cell apoptosis, such as a) ATRA and b) optionally, ATO or azacytidine.

A preferred pharmaceutical combination as defined above comprises:
(i) at least one nucleic acid comprising an antisense and flipped sequence of a fragment of the sequence coding for a kanamycin resistance protein, wherein said nucleic acid is pVAX14 plasmid of sequence SEQ ID NO: 3 and wherein said nucleic acid is formulated with a nano-vehicle, and
(ii) at least one non-immunosuppressive inducer of tumor cell apoptosis, wherein said non-immunosuppressive inducer of tumor cell apoptosis is ATRA, arsenic or ATO or azacytidine.

In one embodiment, the present invention relates to a pharmaceutical combination as defined above comprising:
(i) only one nucleic acid comprising an antisense and flipped sequence of a fragment of the sequence coding for a kanamycin resistance protein, wherein said nucleic acid is pVAX14 plasmid of sequence SEQ ID NO: 3 and wherein said nucleic acid is formulated with a nano-vehicle, and
(ii) only one or two non-immunosuppressive inducer(s) of tumor cell apoptosis, wherein said non-immunosuppressive inducer of tumor cell apoptosis is ATRA, arsenic, ATO or azacytidine,
for example a) ATRA and b) optionally, ATO or azacytidine.

Said at least one nucleic acid (i) and said at least one non-immunosuppressive inducer of tumor cell apoptosis (ii) of the pharmaceutical combination as defined above, are each an active ingredient, preferably provided in a therapeutically effective amount in the same pharmaceutical composition or in separate pharmaceutical compositions.

The pharmaceutical compositions preferably comprise the active ingredient (said at least one nucleic acid (i) and/or said at least one non-immunosuppressive inducer of tumor cell apoptosis (ii)) and at least one physiologically acceptable carrier.

When the pharmaceutical combination as defined above comprises two or at least two nucleic acids (i) as defined above, said nucleic acids may be provided in the same pharmaceutical composition or in different pharmaceutical compositions.

By the expression “the pharmaceutical combination comprises two or at least two nucleic acids”, it is herein meant that said nucleic acids are different, i.e. that they do not have the same nucleic sequence.

When the pharmaceutical combination as defined above comprises two or at least two non-immunosuppressive inducers of tumor cell apoptosis (ii) as defined above, said non-immunosuppressive inducers of tumor cell apoptosis may be provided in the same pharmaceutical composition or in different pharmaceutical compositions.

In one embodiment, said nucleic acid (i) and said non-immunosuppressive inducer of tumor cell apoptosis (ii) of the pharmaceutical combination as defined above are each provided in the form of a separate pharmaceutical composition.

The present invention thus particularly relates to a pharmaceutical combination comprising or consisting of:
a first pharmaceutical composition comprising at least one nucleic acid as defined above formulated with a nano-vehicle and at least one physiologically acceptable carrier,
a second pharmaceutical composition comprising at least one non-immunosuppressive inducer of tumor cell apoptosis as defined above, for example ATRA, and at least one physiologically acceptable carrier and
optionally, a third pharmaceutical composition comprising at least another non-immunosuppressive inducer of tumor cell apoptosis as defined above, for example ATO or azacytidine, and at least one physiologically acceptable carrier.

The present invention thus also relates to a pharmaceutical combination comprising:
a first pharmaceutical composition comprising:
   at least one nucleic acid comprising an antisense and flipped sequence of a fragment of the sequence coding for a kanamycin resistance protein, as defined above, formulated with a nano-vehicle,
   at least one physiologically acceptable carrier, and
a second pharmaceutical composition comprising at least one non-immunosuppressive inducer of tumor cell apoptosis, as defined above, for example ATRA, and at least one physiologically acceptable carrier
optionally, a third pharmaceutical composition comprising at least another non-immunosuppressive inducer of tumor cell apoptosis, as defined above, for example ATO or azacytidine, and at least one physiologically acceptable carrier.

The term “physiologically acceptable carrier” is meant to encompass any carrier, which preferably does not interfere with the effectiveness of the biological activity of the active ingredient and that is preferably not toxic to the host to which is administered. Suitable physiologically acceptable carriers are well known in the art and are described for example in Remington's Pharmaceutical Sciences (Mack Publishing Company, Easton, USA, 1985), which is a standard reference text in this field.

The physiologically acceptable carrier include one or more excipients.

The physiologically acceptable carrier for the pharmaceutical composition comprising at least one nucleic acid as defined above may for example comprise Hepes buffered saline solution (HBSS).

The physiologically acceptable carrier for the non-immunosuppressive inducer of tumor cell apoptosis may for example comprise a copolymer (such as a nano-vehicle), a liposome and/or a buffer.

The pharmaceutical combination as defined above may for example corresponds to a kit. The kit may further comprise instructions for the use in the treatment of cancer or of an infectious disease.

Alternatively, the first, the second and the optional third pharmaceutical compositions may be commercialized separately.

The pharmaceutical composition as defined above comprising said nucleic acid (i) preferably corresponds to a plasmid composition.

The pharmaceutical composition as defined above comprising said nucleic acid (i) preferably comprises from 0.5 mg to 10 mg of nucleic acid, preferably from 0.5 mg to 2 mg of nucleic acid.

The pharmaceutical composition as defined above comprising said non-immunosuppressive inducer of tumor cell apoptosis (ii) preferably comprises from 5 mg to 500 mg of non-immunosuppressive inducer of tumor cell apoptosis, preferably from 10 mg to 200 mg of non-immunosuppressive inducer of tumor cell apoptosis, for example from 10 mg to 100 mg of non-immunosuppressive inducer of tumor cell apoptosis.

The pharmaceutical composition as defined above comprising ATRA (ii) preferably comprises from 5 mg to 100 mg of ATRA, preferably from 5 mg to 50 mg of ATRA, for example from 10 mg to 30 mg of ATRA.

The pharmaceutical composition as defined above comprising azacytidine preferably comprises from 5 mg/ml to 100 mg/ml of azacytidine, more preferably from 10 mg/ml to 50 mg/ml of azacytidine, for example from 20 mg/ml to 30 mg/ml of azacytidine.

The pharmaceutical composition as defined above comprising arsenic or ATO (ii) preferably comprises from 0.2 to 20 mg/ml of arsenic or ATO, more preferably from 0.5 mg/ml to 10 mg/ml of arsenic or ATO, for example from 1 mg/ml to 5 mg/ml of arsenic or ATO.

Each of said nucleic acid (i) and said non-immunosuppressive inducer of tumor cell apoptosis (ii) may be provided in the form of one or more dosage units.

The term “dosage unit” herein refers to a physically discrete unit suitable as unitary dosage, each unit containing a predetermined quantity of active ingredient calculated to produce the desired therapeutic effect.

The pharmaceutical combination as defined above may thus comprise:
one or more dosage units of a first pharmaceutical composition as defined above, wherein said first pharmaceutical composition comprises at least one nucleic acid comprising an antisense and flipped sequence of a fragment of the sequence coding for a kanamycin resistance protein, wherein said nucleic acid is formulated with a nano-vehicle and at least one pharmaceutical acceptable carrier,
one or more dosage units of a second pharmaceutical composition as defined above, wherein said second pharmaceutical composition comprises at least one non-immunosuppressive inducer of tumor cell apoptosis, preferably ATRA, and at least one pharmaceutical acceptable carrier, and
optionally, one or more dosage units of a third pharmaceutical composition as defined above, wherein said third pharmaceutical composition comprises at least another non-immunosuppressive inducer of tumor cell apoptosis, for example ATO or azacytidine, and at least one pharmaceutical acceptable carrier.

The pharmaceutical combination as defined above may comprise at least one other drug, preferably suitable for the treatment of cancer.

By “other drug” or “another drug”, it is herein meant a drug, which is not a nucleic acid comprising an antisense and flipped sequence of a fragment of the sequence coding for a kanamycin resistance protein and which is not a non-immunosuppressive inducer of tumor cell apoptosis.

Said other drug is preferably an anti-cancer drug, such as decitabine or a checkpoint inhibitor.

The checkpoint inhibitor as defined above may for example be selected from the group consisting of a PD-1 inhibitor, a PD-L1 inhibitor, a CD47 inhibitor and a CTLA-4 inhibitor

The checkpoint inhibitor as defined above may be selected from the group consisting of a PD-1 antibody, a fragment of a PD-1 antibody, a PD-L1 antibody, a fragment of a PD-L1 antibody, a CD47 antibody, a fragment of a CD47 antibody, a CTLA-4 antibody and a fragment of a CTLA-4 antibody.

In another embodiment, the pharmaceutical combination as defined above does not comprise any other drug suitable for the treatment of cancer and, preferably, does not comprise any other drug.

In another embodiment, the pharmaceutical combination as defined above does not comprise any checkpoint inhibitor.

### Therapeutic Use

The present invention particularly relates to the pharmaceutical combination as defined above for use as a medicament, in particular for the treatment of cancer or an infectious disease.

The present invention particularly relates to a method of treatment of cancer or an infectious disease, comprising administering to a subject in need thereof the pharmaceutical combination as defined above.

Administration of the pharmaceutical combination as defined above advantageously allows activating innate immune pathway, inflammasome pathway and apoptosis.

When treating cancer, the pharmaceutical combination particularly allows reducing tumor burden and increasing memory T-cells, thereby prolonging remission, preventing relapse and resulting in a durable protection against cancer.

By “treatment of cancer”, it is herein meant a therapeutic use (i.e. on a patient having cancer). The term “treatment” not only includes a treatment leading to complete cure of cancer, but also a treatment slowing down the progression of cancer and/or prolonging the survival of the patient.

As used herein, the term “cancer” refers to any type of malignant (i.e. non benign) tumor.

Cancer may be a solid tumor, preferably selected from the group consisting of breast cancer, colon cancer, a carcinoma, an adenocarcinoma, a sarcoma, a melanoma, a mesothelioma and a blastoma.

Cancer may also be a blood cancer, preferably selected from the group consisting of lymphoid leukaemia, myeloid leukaemia, acute promyelocytic leukaemia (APL), myelodysplastic syndrome (MDS), acute myeloid leukaemia (AML), myelomonocytic leukaemia (CMML), chronic lymphocytic leukaemia (CLL), chronic myelogenous leukaemia (CML), childhood acute lymphoblastic leukaemia (ALL), Hodgkin lymphoma (HL), non-Hodgkin lymphoma (NHL) and multiple myeloma (MM).

When treating an infectious disease, the pharmaceutical combination particularly allows targeting of a viral antigen, in particular with pVAX14 plasmid as nucleic acid formulated with a nano-vehicle and ATRA as non-immunosuppressive inducer of tumor cell apoptosis.

The infectious disease is preferably a viral disease.

The infectious disease may for example be selected from the group consisting of hepatitis, a disease caused by SARS-CoV-2 and a disease caused by the Dengue virus.

By “subject in need thereof”, it is herein meant a subject suffering from or susceptible of suffering from the disease to be treated. The subject to be treated in the frame of the invention is preferably a human being, also referred to as “patient”. However, the veterinary use of the pharmaceutical combination is also contemplated by the present invention. The subject may thus be a human being or a non-human mammal.

Said at least one nucleic acid (i) and said at least one non-immunosuppressive inducer of tumor cell apoptosis (ii) are particularly administered in a therapeutically effective amount.

By “therapeutically effective amount”, it is herein meant an amount sufficient to achieve a concentration of active ingredient, which is capable of preventing, treating or slowing down the disease to be treated. Such concentrations can be routinely determined by those of skilled in the art. The amount of the active ingredient actually administered will typically be determined by a physician, in the light of the relevant circumstances, including the condition to be treated, the chosen route of administration, the actual active ingredient administered, the age, weight, and response of the individual subject, the severity of the subject's symptoms, and the like.

Said at least one nucleic acid (i) and said at least one non-immunosuppressive inducer of tumor cell apoptosis (ii) may be administered simultaneously, i.e. at the same time, or sequentially, i.e. at different times during the course of a common treatment schedule. Said simultaneous or sequential administration may change during the course of treatment.

For example, said nucleic acid (i) and said non-immunosuppressive inducer of tumor cell apoptosis may be administered at the same time at the beginning of the treatment, and sequentially in the following.

In one embodiment, said nucleic acid (i) and said non-immunosuppressive inducer of tumor cell apoptosis (ii) are administered separately at the same time at the beginning of the treatment, and sequentially in the following.

The nucleic acid as defined above may be administered as a single dose, once a week, once every two weeks, once every three weeks or once a month, for example for at least 8 weeks, preferably for at least 3 months, more preferably for at least 4 months.

The nucleic acid as defined above may for example be administered once to four times or at least four times, with an interval of two weeks between two administrations, optionally followed by one or at least two administrations at an interval of one to two months between two administrations.

The non-immunosuppressive inducer of tumor cell apoptosis, in particular ATRA, as defined above, may be administered, preferably orally, once a day, for example for ten days, preferably at the beginning of the treatment, more preferably each time said nucleic acid (i) is administered.

The non-immunosuppressive inducer of tumor cell apoptosis, in particular ATO, as defined above, may be administered, preferably intravenously, for example once a day, for example for ten days, preferably at the beginning of the treatment, more preferably each time said nucleic acid (i) is administered.

The non-immunosuppressive inducer of tumor cell apoptosis, in particular azacytidine, as defined above, may be administered 75 mg per square meter of body-surface area, preferably subcutaneously or intravenously, for example on days 1 through 7 every 28-days, for example once a day, for example for seven days, preferably at the beginning of the treatment, more preferably each time said nucleic acid (i) is administered.

In one embodiment, two non-immunosuppressive inducers of tumor cell apoptosis are administered, such as ATRA and ATO, or ATRA and azacytidine. The two non-immunosuppressive inducers of tumor cell apoptosis may be provided in the same pharmaceutical composition or separately, i.e. in two pharmaceutical compositions. The mode and frequency of administration of these non-immunosuppressive inducers of tumor cell apoptosis is particularly as defined above and below.

Frequency of administration of active ingredient (i) and/or (ii) may vary during the course of treatment. For example, frequencies may be shorter at the beginning of the treatment and then more spaced out during the course of treatment.

When treating cancer, the pharmaceutical combination is preferably administered at least until disappearance of the tumor(s), more preferably until at least one month after disappearance of the tumor(s).

The pharmaceutical combination may also be administered in a stable state of disease or during complete remission, to prevent re-occurrence of the tumor(s).

In one embodiment,
the nucleic acid (i) as defined above is administered once or at least twice (for example from two to four times) with an interval of two weeks between two administrations, optionally followed by one or at least two administrations at an interval of one to two months,
the non-immunosuppressive inducer of tumor cell apoptosis (ii) is preferably ATRA and is administered once a day for 10 days each time said nucleic acid (i) is administered, and/or
a second non-immunosuppressive inducer of tumor cell apoptosis (ii) is used and is ATO or azacytidine.

When treating an infectious disease, the pharmaceutical combination is preferably administered at least until disappearance of the symptoms, for example every two weeks until disappearance of the symptoms.

The amount of nucleic acid (i) to be administered depends, e.g., on the strength of the promoter used, the condition of the mammal intended for administration (e.g., weight or age), the route of administration and the type of formulation.

The nucleic acid as defined above may be administered at a dosage of from 1 μg to 8 mg, preferably from 100 μg to 5 mg, more preferably from 500 μg to 2 mg, in particular for a human adult.

The nucleic acid as defined above may for example be administered at a dosage ranging from 500 μg at the beginning of the treatment to 2 mg at the end of the treatment, in particular for a human adult.

The amount of non-immunosuppressive inducer of tumor cell apoptosis (ii) to be administered depends, e.g. on the condition of the mammal intended for administration (e.g., weight or age), the route of administration, the type of non-immunosuppressive inducer of tumor cell apoptosis and the type of formulation.

The non-immunosuppressive inducer of tumor cell apoptosis may be administered at a dosage of from 20 mg/m² to 150 mg/m², preferably from 30 mg/m² to 100 mg/m², for example from 40 mg/m² to 75 mg/m², preferably once a day for 7 to 10 days, preferably every time said nucleic acid is administered.

The non-immunosuppressive inducer of tumor cell apoptosis ATRA may for example be administered at a dosage of 45 mg/m², preferably once a day orally for 10 days, preferably every time said nucleic acid is administered.

The non-immunosuppressive inducer of tumor cell apoptosis ATO may be administered at a dosage of 0.15 mg/kg, preferably once a day for 10 days, every time said nucleic acid is administered.

The non-immunosuppressive inducer of tumor cell apoptosis azacytidine may be administered at a dosage of 0.15 mg/kg, preferably once a day for 7 days, every time said nucleic acid is administered.

Said nucleic acid (i) and/or said non-immunosuppressive inducer of tumor cell apoptosis (ii) may be administered via an intradermal, intra-epidermal, subcutaneous, intramuscular, intravenous, oral route or intra-tumoral route.

The nucleic acid (i) as defined above is formulated with a nano-vehicle, such as nanotaxi®, in particular as disclosed above.

The route of administration for the nucleic acid (i) as defined above is any conventional route used in the field of DNA vaccination, such as a needle free system (Teixeira et al 2020).

A nucleic acid formulated with a nano-vehicle as defined above, such as nanotaxi®, is preferably administered via intramuscular route. For HR-MDS (Higher-Risk Myelodysplastic Syndromes) patients, who have coagulation problems, the intradermal route is preferably used. Both intramuscular and intradermal routes of administration can be delivered with conventional needle or the needle free system (Teixeira et al 2020).

The route of administration for the non-immunosuppressive inducer of tumor cell apoptosis is preferably oral route, in particular for ATRA, intravenous route, in particular for ATO or azacytidine, or subcutaneously route, in particular for azacytidine.

### Biomarkers of Activation of an Immune Response

The present invention also relates to a panel of 29 biomarkers useful for detecting activation of an immune response.

These biomarkers may advantageously be used to assess activation of an immune response in a biological sample, preferably in a biological sample of a subject treated with the pharmaceutical combination as defined above.

These 29 biomarkers are genes NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2.

A biomarker is referred to in the following by “gene X” or “X”, wherein X is the name of the gene.

The following relates to the human genes as the ultimate targets for this immunotherapy.

NLRP3 gene (also called NLR family pyrin domain containing 3 gene) encodes a protein interacting with the apoptosis-associated speck-like protein PYCARD/ASC, which contains a caspase recruitment domain, and is a member of the NLRP3 inflammasome complex. This complex functions as an upstream activator of NF-kappaB signaling, and it plays a role in the regulation of inflammation, the immune response, and apoptosis.

A reference sequence for said gene is for example HGNC: 16400, NCBI Entrez Gene: 114548, Ensembl: ENSG00000162711, or OMIM®: 606416.

TNFAIP3 gene (Tumor necrosis factor, alpha-induced protein 3) encodes a zinc finger protein and ubiquitin-editing enzyme, and has been shown to inhibit NF-kappa B activation as well as TNF-mediated apoptosis.

A reference sequence for said gene is for example HGNC: 11896, NCBI Entrez Gene: 7128, Ensembl: ENSG00000118503, or OMIM®: 191163.

IL10 (Interleukin 10) gene encodes a protein which is a cytokine that can block NF-kappa B activity. This cytokine has pleiotropic effects in immunoregulation and inflammation. It down-regulates the expression of Th1 cytokines, MHC class II Ags, and costimulatory molecules on macrophages. It also enhances B cell survival, proliferation, and antibody production.

A reference sequence for said gene is for example HGNC: 5962, NCBI Entrez Gene: 3586, Ensembl: ENSG00000136634, or OMIM®: 124092.

KLF4 (Kruppel-like factor 4) gene encodes a protein that belongs to the Kruppel family of transcription factors. The encoded zinc finger protein is required for normal development of the barrier function of skin. The encoded protein is thought to control the G1-to-S transition of the cell cycle following DNA damage by mediating the tumor suppressor gene p53.

A reference sequence for said gene is for example HGNC: 6348, NCBI Entrez Gene: 9314, Ensembl: ENSG00000136826, or OMIM®: 602253.

REL (REL Proto-Oncogene, NF-KB Subunit) gene encodes a protein that belongs to the Rel homology domain/immunoglobulin-like fold, plexin, transcription factor (RHD/IPT) family. Members of this family regulate genes involved in apoptosis, inflammation, the immune response, and oncogenic processes. This proto-oncogene plays a role in the survival and proliferation of B lymphocytes. A reference sequence for said gene is for example HGNC: 9954, NCBI Entrez Gene: 5966, Ensembl: ENSG00000162924, or OMIM®: 164910.

TREM1 (Triggering receptor expressed on myeloid cells 1) gene encodes a receptor belonging to the Ig superfamily that is expressed on myeloid cells. This protein amplifies neutrophil and monocyte-mediated inflammatory responses triggered by bacterial and fungal infections by stimulating release of pro-inflammatory chemokines and cytokines, as well as increased surface expression of cell activation markers.

A reference sequence for said gene is for example HGNC: 17760, NCBI Entrez Gene: 54210, Ensembl: ENSG00000124731, or OMIM®: 605085.

RNF19B (Ring Finger Protein 19B) gene encodes a multi-pass membrane protein containing two RING-type and one IBR-type zinc finger motifs. The encoded protein is an E3 ubiquitin-protein ligase that plays a role in the cytotoxic effects of natural killer (NK) cells.

A reference sequence for said gene is for example HGNC: 26886, NCBI Entrez Gene: 127544, Ensembl: ENSG000001 16514, or OMIM®: 610872.

TRIM25 (Tripartite Motif Containing 25) gene encodes a protein which is a member of the tripartite motif (TRIM) family. The TRIM motif includes three zinc-binding domains, a RING, a B-box type 1 and a B-box type 2, and a coiled-coil region. The protein is an RNA binding protein, functions as an ubiquitin E3 ligase and is involved in multiple cellular processes, including regulation of antiviral innate immunity.

A reference sequence for said gene is for example HGNC: 12932, NCBI Entrez Gene: 7706, Ensembl: ENSG00000121060, or OMIM®: 600453.

ZC3H12A (Zinc Finger CCCH-Type Containing 12A) is a Protein Coding gene. It acts as a transcriptional activator and causes cell death of cardiomyocytes, possibly via induction of genes associated with apoptosis.lt functions as an endoribonuclease involved in mRNA decay and involved in various biological functions such as cellular inflammatory response and immune homeostasis, glial differentiation of neuroprogenitor cells, cell death of cardiomyocytes, adipogenesis and angiogenesis.

A reference sequence for said gene is for example HGNC: 26259, NCBI Entrez Gene: 80149, Ensembl: ENSG00000163874, or OMIM®: 610562.

CSF1 (ColonySstimulating Factor 1)—FMS gene encodes a cytokine that controls the production, differentiation, and function of macrophages. This cytokine plays an essential role in the regulation of survival, proliferation and differentiation of hematopoietic precursor cells, especially mononuclear phagocytes, such as macrophages and monocytes. It promotes the release of pro-inflammatory chemokines, and thereby plays an important role in innate immunity and in inflammatory processes.

A reference sequence for said gene is for example HGNC: 2432, NCBI Entrez Gene: 1435, Ensembl: ENSG00000184371, or OMIM®: 120420.

CXADR (CXADR Ig-Like Cell Adhesion Molecule) gene encodes a protein of a type I membrane receptor for group B coxsackieviruses and subgroup C adenoviruses. It is involved in transepithelial migration of leukocytes through adhesive interactions with JAML a transmembrane protein of the plasma membrane of leukocytes. The interaction between both receptors also mediates the activation of gamma-delta T-cells, a subpopulation of T-cells residing in epithelia and involved in tissue homeostasis and repair. Upon epithelial CXADR-binding, JAML induces downstream cell signaling events in gamma-delta T-cells through PI3-kinase and MAP kinases. It results in proliferation and production of cytokines and growth factors by T-cells that in turn stimulate epithelial tissues repair.

A reference sequence for said gene is for example HGNC: 2559, NCBI Entrez Gene: 1525, Ensembl: ENSG00000154639, or OMIM®: 602621.

HIST1H2BC (H2BC4) (H2B Clustered Histone 4) gene encodes a histone protein, a nuclear protein responsible for the nucleosome structure of the chromosomal fiber in eukaryotes. The protein has antibacterial and antifungal antimicrobial activity.

A reference sequence for said gene is for example HGNC: 4757, NCBI Entrez Gene: 8347, Ensembl: ENSG00000180596, or OMIM®: 602847.

JAG1 or JAGGED1 is the ligand for the receptor notch 1, involved in signaling processes that finctions to establish and regulate cell fate.

A reference for said gene is for example HGNC: 6188, NCBI Entrez Gene: 182, Ensembl: ENSG00000101384 or OMIM®: 601920.

KDM6B (Lysine Demethylase 6B) gene encodes lysine-specific demethylase that specifically demethylates di- or tri-methylated lysine 27 of histone H3 (H3K27me2 or H3K27me3). H3K27 trimethylation is a repressive epigenetic mark controlling chromatin organization and gene silencing. It is involved in inflammatory response by participating in macrophage differentiation in case of inflammation by regulating gene expression and macrophage differentiation

A reference sequence for said gene is for example HGNC: 29012, NCBI Entrez Gene: 23135,Ensembl: ENSG00000132510 MIM®: 611577, or OMIM®: 611577.

NFKBID (NFKB Inhibitor Delta) is a Protein Coding gene. It is predicted to enable NF-kappaB binding activity, predicted to be involved in T cell receptor signaling pathway, positive regulation of T-helper 17 cell differentiation and regulation of gene expression. It is predicted to act upstream of or within several processes, including negative regulation of NF-kappaB transcription factor activity, negative regulation of T cell differentiation in thymus, and positive regulation of thymocyte apoptotic process.

A reference sequence for said gene is for example HGNC: 15671, NCBI Entrez Gene: 84807, Ensembl: ENSG00000167604, or OMIM®: 618887.

OLR1gene (Oxidized low-density lipoprotein receptor 1) encodes a low density lipoprotein receptor that belongs to the C-type lectin superfamily. This receptor mediates the recognition, internalization and degradation of oxidatively modified low density lipoprotein (oxLDL) by vascular endothelial cells. OxLDL is a marker of atherosclerosis that induces vascular endothelial cell activation and dysfunction, resulting in pro-inflammatory responses, pro-oxidative conditions and apoptosis. Its association with OxLDL induces the activation of NF-kappa-B through an increased production of intracellular reactive oxygen and a variety of pro-atherogenic cellular responses including a reduction of nitric oxide (NO) release, monocyte adhesion and apoptosis.

A reference sequence for said gene is for example HGNC: 8133, NCBI Entrez Gene: 4973, Ensembl: ENSG00000173391, or OMIM®: 602601.

PMAIP1 gene (Phorbol-12-Myristate-13-Acetate-Induced Protein 1) belongs to a pro-apoptotic subfamily within the BCL-2 protein family. It promotes activation of caspases and apoptosis.

A reference sequence for said gene is for example HGNC: 9108, NCBI Entrez Gene: 5366, Ensembl: ENSG00000141682, or OMIM®: 604959.

SELP (Selectin P) gene encodes a 140 kDa protein that is stored in the alpha-granules of platelets and Weibel-Palade bodies of endothelial cells. This protein mediates rapid rolling of leukocyte rolling over vascular surfaces during the initial steps in inflammation through interaction with SELPLG. Cell adhesion molecules (CAMs) are a large family of transmembrane proteins that are involved in the binding of a cell to another cell or to the extracellular matrix. They have roles in cell proliferation, differentiation, motility, trafficking, apoptosis and tissue architecture.

A reference sequence for said gene is for example HGNC: 10721, NCBI Entrez Gene: 6403, Ensembl: ENSG00000174175, or OMIM®: 173610.

SRGN (Serglycin) gene encodes a protein best known as a hematopoietic cell granule proteoglycan. This encoded protein was found to be associated with the macromolecular complex of granzymes and perforin, which may serve as a mediator of granule-mediated apoptosis.

A reference sequence for said gene is for example HGNC: 9361. NCBI Entrez Gene: 5552, Ensembl: ENSG00000122862, or OMIM®: 177040.

NFKBIZ(NF-kappa-B inhibitor zeta) gene is a member of the ankyrin-repeat family and is induced by lipopolysaccharide (LPS). It is Involved in regulation of NF-kappa-B transcription factor complexes. The C-terminal portion of the encoded product contains the ankyrin repeats, shares high sequence similarity with the I kappa B family of proteins. The latter are known to play a role in inflammatory responses to LPS by their interaction with NF-B proteins through ankyrin-repeat domains.

A reference sequence for said gene is for example HGNC: 29805, NCBI Entrez Gene: 64332, Ensembl: ENSG00000144802, or OMIM®: 608004.

PER1 is the Period Circadian Regulator 1. It encodes the protein which acts as a transcriptional repressor, which forms a core component of the circadian clock, an internal time-keeping system. Polymorphisms in this gene may increase the risk of getting certain cancers.

A reference for said gene is for example HGNC: 8845, NCBI Entrez Gene: 5187, Ensembl: ENSG00000179094 or OMIM®: 602260.

AREG encodes a protein of the epidermal growth factor (EGF) family. It is an autocrine growth factor and mitogen for astrocytes, Schwann cells and fibroblasts. The protein interacts with the EGF//TGF-alpha receptor to promote the growth of normal epithelial cells and inhibits the growth of aggressive carcinoma cells. It also functions as mammary gland, oocyte and bone tissue development.

A reference for said gene is for example HGNC: 651, NCBI Entrez Gene: 374, Ensembl: ENSG00000109321 or OMIM®: 104640.

ATF4 encodes a activating transcription factor 4 originally identified as a mammalian DNA binding protein that could bind a tax-responsive enhancer element in the LTR of HTLV-1. Its is also known as the cAMP responsive element binding protein 2 (CREB-2).

A reference for said gene is for example HGNC: 786, NCBI Entrez Gene: 468, Ensembl: ENSG00000128272 or OMIM®: 604064.

BTG1 encodes an anti-proliferation factor 1 and the family regulates growth and differentiation. Together with several nuclear receptors, its functions as a coactivator of cell differentiation.

A reference for said gene is for example HGNC: 1130, NCBI Entrez Gene: 694, Ensembl: ENSG00000133639 or OMIM®: 109580.

HMGCS1 for 3-Hydroxy-3-Methylglutaryl-CoA Synthase 1 enables homodimerisaton Activity. It is predicted to be involved in acetyl-CoA metabolism and farnesyl diphosphate biosynthesis and mevalonate pathway.

A reference list for said gene is for example HGNC: 5007, NCBI Entrez Gene: 3157, Ensembl: ENSG00000112972 or OMIM®: 142940.

JMJD6 (Jumonji Domain Containing 6, Arginine Demethylase And Lysine Hydroxylase) is predicted to function as protein hydrolase or histone demethylase. It was originally thought to be involved in phagocytosis of apoptotic cells, but may not be directly involved in clearance of apoptotic cells.

A reference list for said gene is for example HGNC: 19355, NCBI Entrez Gene: 23210, Ensembl: ENSG00000070495 or OMIM®: 604914 MAP3K8 for Mitogen-Activated Protein Kinase Kinase Kinase 8 is an oncogene that encodes a member of the serine/threonine protein kinase family. Cytoplasmic in location, it can activate MAP kinase and JNK kinase pathways. It activates the iKappaB kinases and induces nuclear production of NFKappaB, which promotes the production of TNF-αlha and IL-2 during T-cell activation.

A reference list for said gene is for example HGNC: 6860, NCBI Entrez Gene: 1326, Ensembl: ENSG00000107968 or OMIM®: 191195.

NR4A1 for Nuclear Receptor Subfamily 4 Group A Member 1 is a member of the steroid-thyroid hormone-retinoid receptor superfamily. It is a nuclear transcription factor.

Translocation from nucleus to mitochondria induces apoptosis.

A reference list for said gene is fro example HGNC: 7980, NCBI Entrez Gene: 3164, Ensembl: ENSG00000123358 or OMIM®: 139139.

NR4A2 for Nuclear Receptor Subfamily 4 Group A member 2 is a member of the steroid-thyroid hormone-retinoid receptor superfamily and may act a transcription factor. It is important for differentiation and maintenance of neurons during development.

A reference list for said gene is for example HGNC: 7981, NCBI Entrez Gene: 4929, Ensembl: ENSG00000153234 or OMIM®: 601828.

### Method for Assessing Activation of an Immune Response

The present invention also relates to a method for assessing activation of an immune response, wherein said method comprises:
a) measuring the quantity of at least 5 biomarkers selected from the group consisting of NLRP3, TNFAIP3, IL10, KLF4, REL, TREM1, RNF19b, TRIM25, ZCH12A, CSF1-FMS, CXADR, HIST1H2BC, JAG1, KDM6B, NFKBID, OLR1, PMAIP1, SELP, SRGN, NFKBIZ, PER1, AREG, ATF4, BTG1, HMGCS1, JMJD6, MAP3K8, NR4A1 and NR4A2 in a biological sample,
b) optionally, comparing the results obtained in step a) with a corresponding control value, and
c) optionally, deducing if an activation of an immune response is present in the biological sample.

### Step a)

In step a), the quantity of at least 5 biomarkers selected from the group consisting of NLRP3, TNFAIP3, IL10, KLF4, REL, TREM1, RNF19b, TRIM25, ZCH12A, CSF1-FMS, CXADR, HIST1H2BC, JAG1, KDM6B, NFKBID, OLR1, PMAIP1, SELP, SRGN, NFKBIZ, PER1, AREG, ATF4, BTG1, HMGCS1, JMJD6, MAP3K8, NR4A1 and NR4A2 is measured in a biological sample.

Step a) may comprise measuring the quantity of at least 5 biomarkers, preferably at least 7 biomarkers, at least 10 biomarkers, at least 12 biomarkers, at least 14 biomarkers or at least 16 biomarkers, more preferably at least 19 biomarkers or at least 22 biomarkers, still more preferably at least 25 biomarkers, selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2 genes, in a biological sample.

Step a) may comprise measuring the quantity of at least 5 biomarkers, preferably at least 7 biomarkers, at least 10 biomarkers, at least 12 biomarkers, at least 14 biomarkers or at least 16 biomarkers, selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP and SRGN.

Step a) may for example comprise measuring the quantity of biomarkers NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN and, optionally, of at least one biomarker (or at least two, at least four, at least six, at least eight or at least ten biomarkers) selected from the group consisting of NFKBIZ, PER1, AREG, ATF4, BTG1, HMGCS1, JMJD6, MAP3K8, NR4A1 and NR4A2.

In one embodiment, in step a), the quantity of biomarkers NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2 is measured in a biological sample.

The quantity of a biomarker is preferably the level of mRNA expression.

The level of mRNA expression may be measured by any method well-known by the skilled person, such as by RQ-PCR (real-time quantitative reverse transcriptase polymerase chain reaction).

The biological sample may be a blood sample, for example a peripheral blood sample, or a tissue sample, for example a bone marrow sample.

By “peripheral blood”, it is herein meant the blood circulating throughout the body, and not concentrated within a specific organ.

The blood sample is preferably obtained by collecting blood in capillary tubes comprising EDTA as an anticoagulant.

The bone marrow sample is for example obtained by flushing the marrow from long bones into a culture medium, preferably comprising 2% of serum. The bone marrow sample may be frozen for later use.

The biological sample preferably does not consist of peripheral blood purified exosomes. More generally, the biological sample preferably does not consist of blood-derived purified exosomes, more preferably does not consist of exosomes.

The biological sample preferably does not consist of Type 2 innate lymphoid cells (ILC2s).

Measuring the quantity of said biomarkers, in particular the level of mRNA expression of said biomarkers, may be carried out directly in a biological sample from a subject.

For example, the peripheral blood sample may be directly used for RNA extraction, for example by mixing the peripheral blood sample with trizol.

For example, the bone marrow sample may be used directly for RNA extraction.

The subject may be a human being or a non-human animal, for example a non-human mammal.

Said subject preferably suffers from cancer or from an infectious disease.

Said subject may be a treated subject.

By “treated subject”, it is herein meant a subject treated with a nucleic acid, such as at least one nucleic acid as defined above comprising an antisense and flipped sequence of a fragment of the sequence coding for a kanamycin resistance protein, wherein said nucleic acid is capable of eliciting an immune response in a mammal, preferably in combination with at least one non-immunosuppressive inducer of tumor cell apoptosis.

The treated subject is preferably treated with the pharmaceutical combination as defined above.

### Step b)

In step b), the results obtained at step a) are compared with a corresponding control value.

The term “corresponding” in the expression “corresponding control value” means that, for each biomarker, its quantity is compared to the control value of said biomarker.

A control value is for example the mean, maximal value and/or minimal value of the quantity of biomarker measured in samples from a reference population, for example from non-treated diseased subjects.

Alternatively, the control value may be the quantity of biomarker in a sample from the same subject, but before treatment or at a previous time during the course of treatment.

### Step c)

In step c), it is deduced if an activation of an immune response is present in the biological sample.

In particular, there is an activation of an immune response if all the measured biomarkers are upregulated by comparison to the corresponding control value.

A biomarker is upregulated if its measured quantity is higher than the corresponding control value.

Said control value is preferably a pre nucleic acid treatment value, i.e. a value measured before nucleic acid treatment.

### Method for Monitoring Response to a Treatment

The present invention also relates to a method for monitoring response to a treatment, in particular response to a treatment aiming at activating an immune response, such as a treatment based on the pharmaceutical combination as defined above.

The method as defined above for monitoring response to a treatment is an in vitro method.

The method as defined above for monitoring response to a treatment in a subject particularly comprises:
a) measuring the quantity, preferably the level of mRNA expression, of at least 5 biomarkers selected from the group consisting of NLRP3, TNFAIP3, IL10, KLF4, REL, TREM1, RNF19b, TRIM25, ZCH12A, CSF1-FMS, CXADR, HIST1H2BC, JAG1, KDM6B, NFKBID, OLR1, PMAIP1, SELP, SRGN, NFKBIZ, PER1, AREG, ATF4, BTG1, HMGCS1, JMJD6, MAP3K8, NR4A1 and NR4A2 in a biological sample from said subject,
b) comparing the results obtained at step a) with a corresponding control value and/or to a corresponding value from said subject measured before beginning the treatment or measured in the course of treatment,
c) deducing if the subject responds favorably to the treatment, and
d) optionally, repeating steps a) to c).

Said treatment is preferably a treatment comprising administering a nucleic acid, such as at least one nucleic acid as defined above comprising an antisense and flipped sequence of a fragment of the sequence coding for a kanamycin resistance protein, wherein said nucleic acid is capable of eliciting an immune response in a mammal, preferably in combination with at least one non-immunosuppressive inducer of tumor cell apoptosis.

Said treatment is preferably a treatment comprising administering the pharmaceutical combination as defined above, in particular as disclosed above in the section “Therapeutic use”.

The subject as defined above preferably suffers from cancer or from an infectious disease.

### Step a)

Step a) may comprise measuring the quantity, preferably the level of mRNA expression, of at least 5 biomarkers, preferably at least 7 biomarkers, at least 10 biomarkers, at least 12 biomarkers, at least 14 biomarkers or at least 16 biomarkers, more preferably at least 19 biomarkers or at least 22 biomarkers, still more preferably at least 25 biomarkers, selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2, in a biological sample.

Step a) may comprise measuring the quantity of at least 5 biomarkers, preferably at least 7 biomarkers, at least 10 biomarkers, at least 12 biomarkers, at least 14 biomarkers or at least 16 biomarkers, selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP and SRGN.

Step a) may for example comprise measuring the quantity, preferably the level of mRNA expression, of biomarkers NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN and, optionally, of at least one biomarker (or at least two, at least four, at least six, at least eight or at least ten biomarkers) selected from the group consisting of NFKBIZ, PER1, AREG, ATF4, BTG1, HMGCS1, JMJD6, MAP3K8, NR4A1 and NR4A2.

In one embodiment, in step a), the quantity of biomarkers NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2 is measured in a biological sample from said subject.

Step a) may be carried out as step a) defined above in the section “Method for assessing activation of an immune response”.

The sample is particularly as defined above in the section “Method for assessing activation of an immune response”.

The sample is preferably obtained from said subject at least two weeks after the beginning of treatment or at least one month after the beginning of treatment, such as for example one month, two months, three months or at least four months after the beginning of treatment.

### Step b)

In optional step b), the results obtained at step a) are compared with a corresponding control value and/or to a corresponding value from said subject measured before beginning the treatment or measured in the course of treatment.

The corresponding control value is particularly as defined in step b) defined above in the section “Method for assessing activation of an immune response”.

The corresponding value from said subject measured before beginning the treatment or measured in the course of treatment is a value measured in a biological sample obtained from said subject before beginning the treatment or in the course of treatment, i.e. at a previous time during the course of treatment, respectively.

### Step c)

In optional step c), it is deduced if the subject responds favorably to the treatment.

In particular, the subject responds favorably to the treatment if an activation of the immune response is detected in the biological sample.

In particular, there is an activation of an immune response if all the measured biomarkers are upregulated by comparison to the corresponding control value or by comparison to the corresponding value from said subject measured before beginning the treatment or measured in the course of treatment.

Said control value is preferably a pre nucleic acid treatment value, i.e. a value measured before nucleic acid treatment.

If the subject does not reply favorably to treatment, a different treatment may be administered.

### Optional Step d)

In optional step d), steps a) to c) are repeated.

Steps a) to c) may be repeated several times during the course of treatment.

For example, steps a) to c) may first be carried out on a sample obtained from said subject 2 weeks after the beginning of treatment, and then steps a) to c) may be repeated monthly, for example on a sample obtained from said subject one month after the beginning of treatment, then two months after the beginning of treatment, then three months after the beginning of treatment, etc.

Step d) may be carried out even if the subject does not respond favorably to treatment in step b).

### Kit and Use of Said Kit

The present invention also relates to a kit suitable for assessing activation of an immune response, wherein said kit comprises means for detecting at least 5 biomarkers, preferably at least 7 biomarkers, at least 10 biomarkers, at least 12 biomarkers, at least 14 biomarkers or at least 16 biomarkers, more preferably at least 19 biomarkers or at least 22 biomarkers, still more preferably at least 25 biomarkers selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2.

The kit as defined above may comprise means for detecting biomarkers NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN and, optionally, of at least one biomarker (or at least two, at least four, at least six, at least eight or at least ten biomarkers) selected from the group consisting of NFKBIZ, PER1, AREG, ATF4, BTG1, HMGCS1, JMJD6, MAP3K8, NR4A1 and NR4A2.

The kit as defined above may comprise means for detecting biomarkers NLRP3, TNFAIP3, IL10, KLF4, REL, TREM1, RNF19b, TRIM25, ZCH12A, CSF1-FMS, CXADR, HIST1H2BC, JAG1, KDM6B, NFKBID, OLR1, PMAIP1, SELP, SRGN, NFKBIZ, PER1, AREG, ATF4, BTG1, HMGCS1, JMJD6, MAP3K8, NR4A1 and NR4A2.

The kit as defined above is particularly suitable for carrying out a method for assessing activation of an immune response as defined above or a method for monitoring response to a treatment as defined above.

The means for detecting said biomarkers particularly allow measuring the quantity of said biomarkers, in particular the level of mRNA expression of said biomarkers, for example by RQ-PCR.

The means for detecting said biomarkers for example comprise:
primers for amplifying each of said biomarkers, in particular for amplifying the cDNA of each of said biomarkers,
optionally, at least one reverse transcriptase,
optionally, at least one polymerase,
optionally, at least one buffer, and
optionally, dNTPs (deoxynucleoside triphosphates).

The present invention also relates to the use of the kit as defined above for assessing activation of an immune response, in particular for carrying out a method for assessing activation of an immune response as defined above or a method for monitoring response to a treatment as defined above.

All references cited herein, including journal articles or abstracts, published patent applications, issued patents or any other references, are entirely incorporated by reference herein, including all data, tables, figures and text presented in the cited references.

The invention will be further illustrated in the following examples and figures.

### BRIEF DESCRIPTION OF THE SEQUENCES

SEQ ID NO: 1 corresponds to an antisense and flipped sequence of a fragment of the sequence coding for the kanamycin resistance protein.

SEQ ID NO: 2 corresponds to the flipper region consisting of an antisense and flipped sequence of a fragment of the sequence coding for the kanamycin resistance protein and of an antisense and flipped sequence of a fragment of the pVAX1 plasmid.

SEQ ID NO: 3 corresponds to the complete sequence of the pVAX14 plasmid comprising the flipper region of sequence SEQ ID NO: 2.

SEQ ID NO: 4 corresponds to the sequence of the immunogenic polypeptide encoded by ORF1.

SEQ ID NO: 5 corresponds to the sequence of the immunogenic polypeptide encoded by ORF2.

SEQ ID NO: 6 corresponds to the sequence of the immunogenic polypeptide encoded by ORF5.

SEQ ID NO: 7 corresponds to the sequence of pVAX1 plasmid.

### BRIEF DESCRIPTION OF THE DRAWINGS

FIG. 1. Nanotaxi® and plasmid DNA are adjuvants and activate the innate immune pathway.
A) DNA delivered in Nanotaxi® increases intracytoplasmic DNA sensor (DAI) expression. DAI expression were determined by RT-qPCR analysis. Total RNA was extracted from tissues with TRIzol® (Life technologies) according to the manufacturer's protocol. RT-qPCR were then realized in 2 phases: first, reverse transcriptions of RNA in cDNA were conducted using High-Capacity cDNA Reverse Transcription Kits (Life technologies) on GeneAmp® PCR System 9700 Thermal Cycler (Life technologies). Then, Real-time PCR were effectuated using Taqman® Gene expression assays on StepOnePlus™ Real-Time PCR System (Life technologies). Probes used to amplify specific gene products from murin cDNA (Life technologies) were: for DAI: Mm00457979_m1; for HPRT (Housekeeping gene): Mm00446966_m1.
B) PML-RARA transcript measurement by RQ-PCR as described in Pokorna et al 2013. pVax14 in combination with ATRA or pVAX delivered in Nanotaxi® in combination with ATRA (5 mg 21-day release).

FIG. 2. Increased expression of transcripts of A) high mobility box protein 1 (Hmgb1); B), the adapter molecule MyD88; C) ATRA inducible Rig-I when using pVAX14+ATRA or PMLRARAFC+ATRA plasmid. Controls: pVAX1+ATRA or placebo+ATRA

FIG. 3: Activation of the inflammasome pathway: pVax14+ATRA activates the inflammasome pathway when effectors (E) from immunized AML mice are co-cultured with their leukemia bone marrow (BM) targets (T) at an E;T ratio of 2:1 compared with normal healthy FVB/N BM with A) increased expression of Nlrp3; B) the increase in apoptosis measured using labelled annexin V and the Incucyte as described previously (Gorombei et al. 2021); C) increased Caspase 1 detection by western blot analyses as described previously (Omidvar et al. 2007; Gorombei et al 2021) after 1 or 6 hours of co-culture.

FIG. 4:
A) Protocol of AML 3 transplanted mice treated with placebo+ATRA (5 mg 21-day release pellet)+arsenic trioxide (ATO) (5 g/g/mouse) administered intraperitoneally for 28 days starting on day 6 (D6) or pVAX14 DNA (2×50 μg) administered intramuscularly on day 7 and every 20 days for a total of 3 cycles+ATRA+ATO;
B) Kaplan-Meier survival curves of pVAX14+ATO+ATRA versus ATO+ATRA versus placebo;
C) Count data was imported into Partek Genomics Suite to perform Hierarchical Clustering, Principle Component Analysis and differential expression based ANOVA. Principle component analyses (PCA) showing the tight clustering of gene expression in pVAX14 treated mice (n=7, circled) compared to mice without plasmid treatment (n=5, not circled);
D) Waterfall plot showing some of the genes regulated.
E) Waterfall Plot showing the regulation of the 29 genes of Table 2, from the 16 immune related pathways (Table 1).

FIG. 5:
A) Protocol of Transgenic HR-MDS mice treated with azacytidine (AZA) (IP 3 times per week, 0.5 mg/kg) followed by pVAX14 (50 mg ID weekly for 6 weeks+ATRA (5 mg 21-day release pellet) with ongoing AZA administration.
B) Kaplan-Meier survival curves of AZA followed by pVAX14+ATRA versus Aza alone or placebo.
C) Peripheral blood memory T-cells (percentage of CD44 hi/CD62LIo population within the CD4+) on day 56 showing the highest levels in the AZA followed by pVAX14+ATRA treated group.

FIG. 6: Increased expression of Myd88 in peripheral blood of normal rats when pVAX14 is delivered with Nanotaxi®. Group 1: control; Group 2: Nanotaxi®; Group 3: pVAX14; Group 4: pVAX14+Nanotaxi®.

### EXAMPLE

### PVAX14 Delivery in Nanotaxi® Increases Uptake of DNA and Improves Reduction of Tumor Burden with Activation of the Innate Immune Pathway

### Material and Methods

Formulation of pVAX14 in Nanotaxi®

The nanotaxi® used in the Example is the compound of the following formula:

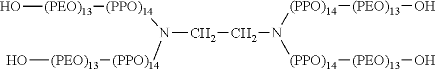

PVAX14 was formulated by mixing equal volumes of Nanotaxi® stock solution in 2× buffer with pVAX14 aqueous solution at the desired concentration, immediately prior to intramuscular injection. The mixing of Nanotaxi® and plasmid PVAX14 is a self-assembly process that results from hydrogen bonding, hydrophobic and electrostatic interactions between Nanotaxi® and DNA.

### RNA Isolation, Library Preparation and Analysis

Total bone marrow samples (comprising precursors of T-cells, B-cells, leukocytes, NL cells, platelets (megakaryocytes) and erythrocytes (erythroblasts)) were harvested by flushing the marrow from long bones into culture media 2% sera and frozen in 20% DMSO at approximately 10⁶ per m1 in heat inactivated foetal Bovine serum. Living cells were cooled slowly in a Mister frosty container which is has 70% methanol to enable slow cooling at −80 degrees. The frozen cells were then transferred into liquid nitrogen for long term storage. For RNA extraction, the cells were first defrosted and 10 m1 media added to 1 m1 vial of cells. Cells were centrifuged and washed in phosphate buffered saline. RNA was then extracted using the Direct-zol RNA Miniprep Kit from Zymo Research as per the manufacturer's instructions.

Following isolation of total RNA, each sample was assessed for RNA integrity using the Agilent BioAnalyser 2100 and the RNA 6000 Nano Kit. Samples with RIN greater than 7 were retained for downstream use.

RNA samples were quantified using a Qubit RNA Broad Range kit and 800 ng of total RNA was retained for library preparation.

Library preparation was performed with 800 ng input total RNA using the NEBNext Ultra II Directional RNA Library Prep kit and NEBNext Poly(A) mRNA Magnetic Isolation Module. Libraries were purified using KAPA Pure beads to select for fragments between 150-700 bp in size. Libraries were sequenced at the Genomics Core Technology Unit, Queens University Belfast using a NextSeq 500.

FASTQ files were downloaded from BaseSpace sequencing hub and checked for quality using FASTQC. Reads were aligned to mm10 using STAR and Ensemble gene annotation files. Gene count data generated by STAR was used as input to DESeq2 to generate differential expression data as well as normalised count tables.

Count data was imported into Partek Genomics Suite to perform Hierarchical Clustering, Principle Component Analysis and differential expression based ANOVA.

Pathway analyses was undertaken using the DAVID software using the Anova with p=0.01 gene list to generate the enrichment using the top 100 genes (data not shown).

pVAX14 in Nanotaxi® in Normal Rats Increase Myd88 Expression

A preliminary toxicity study has been conducted in Wistar rats with a 2-week preliminary intramuscular injection of (i) Hepes buffered saline (HBSS) alone as a control, (ii) Nanotaxi®, (iii) pVAX14 in HBBS (1 mg DNA/animal/injection) or (iv) pVAX14 formulated in Nanotaxi® (1 mg DNA/animal/injection) (twice weekly on days 0, 4, 8 and 12). Injection sites were observed for all animals before and approximately 24 hours after each administration (i.e. on days 1, 5, 9 and 13) in order to evaluate the local tolerance (presence or absence of erythema, oedema, induration, haematoma, swelling, tumefaction, redness). Animals were observed twice daily. On treatment days, the animals were observed before and at least once after dosing to detect any clinical signs or reactions to treatment. No toxicity or adverse effects were found with either pVAX14 or Nanotaxi® alone or together (data not shown). Peripheral blood was collected in capillary tubes with EDTA as an anticoagulant. Peripheral blood (100 μL) was added to 900 μL of Trizol and mixed slowly by pipetting. Peripheral blood RNA was then extracted with an RNA extraction kit according to manufacturer's protocol. Myd88 mRNA expression was then measured by RQ-PCR.

### Results

DNA delivered in Nanotaxi® allows increasing the uptake of DNA (see FIG. 1A).

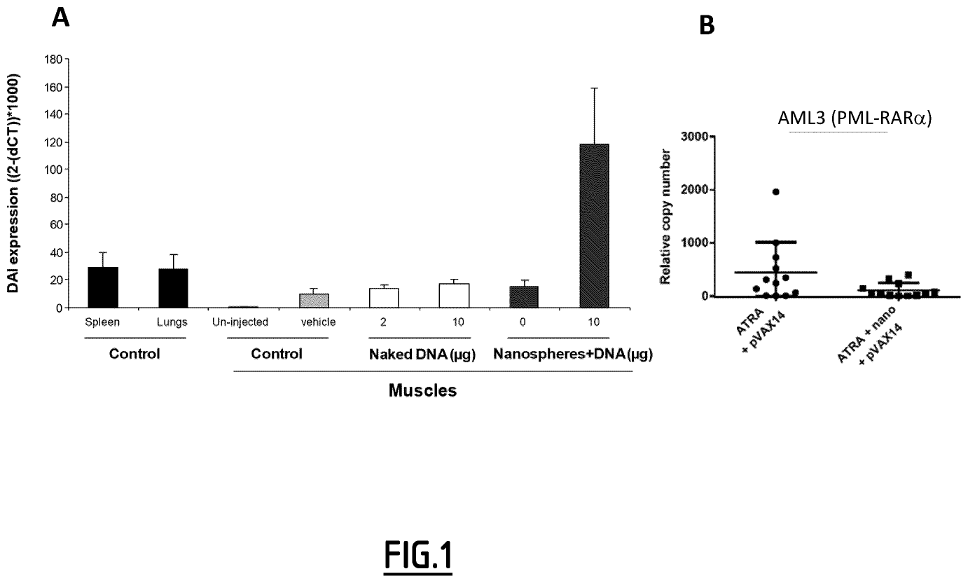

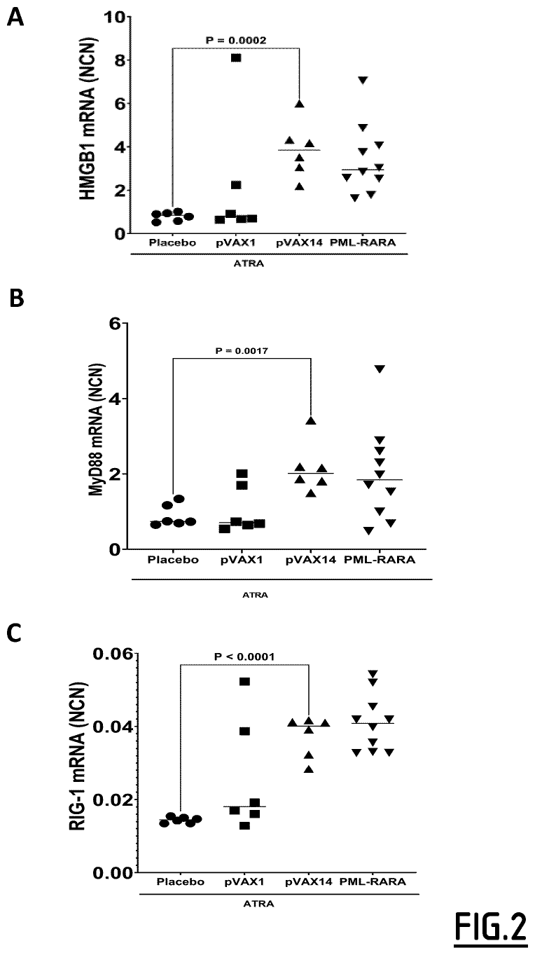

pVax14 delivered in Nanotaxi® in combination with ATRA (5 mg 21-day release) reduces more efficiently the tumor burden, as determined by minimal residual disease assessment (MRD) of the PML-RARA transcript measurement by RQ-PCR (see FIG. 2B).

pVAX14+ATRA activates the innate immune pathway to a similar extent as the specific PMLRARAFC plasmid with increases in transcripts of high mobility box protein 1 (Hmgb1), the adapter molecule MyD88 and ATRA inducible Rig-I (see FIGS. 2A, B and C).

pVax14 delivered in Nanotaxi® synergistically activates the innate immune system pathway in normal rats compared to pVAX14 or Nanotaxi® taken alone, as shown by an increased level of transcription of the adapter molecule MyD88 (see FIG. 6). MYD88 is downstream of TLR9, of which ligand is CpG DNA. Therefore, this is likely to be a DNA-mediated response.

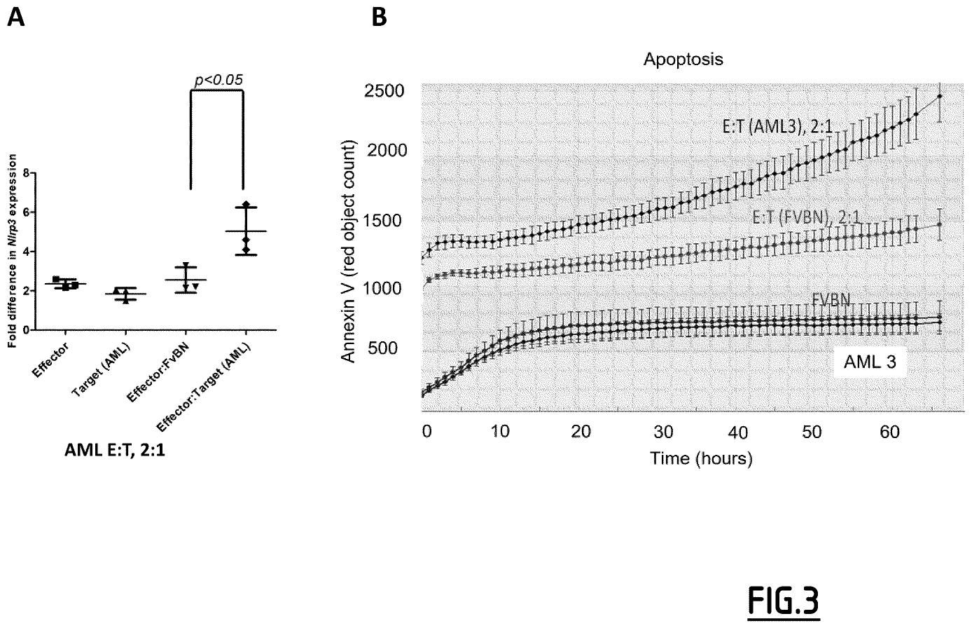

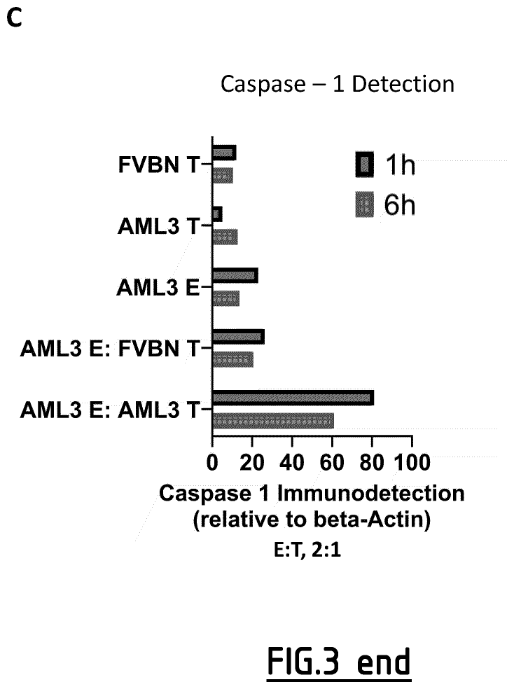

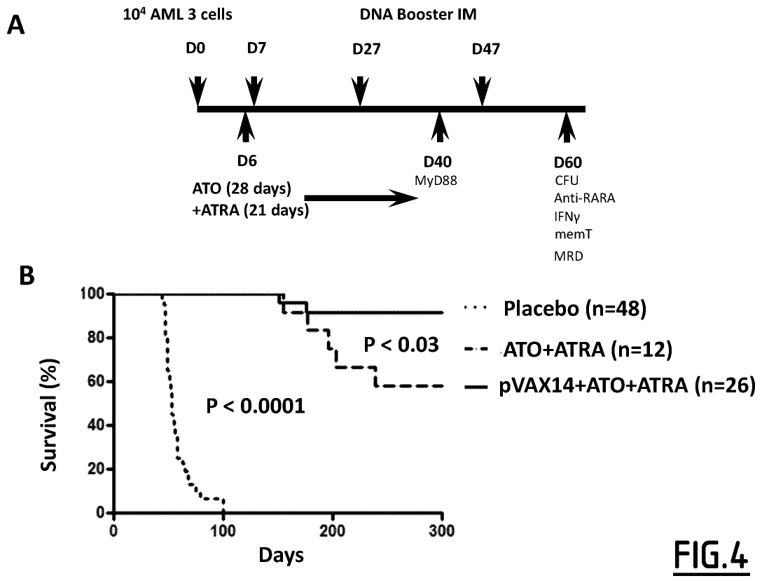

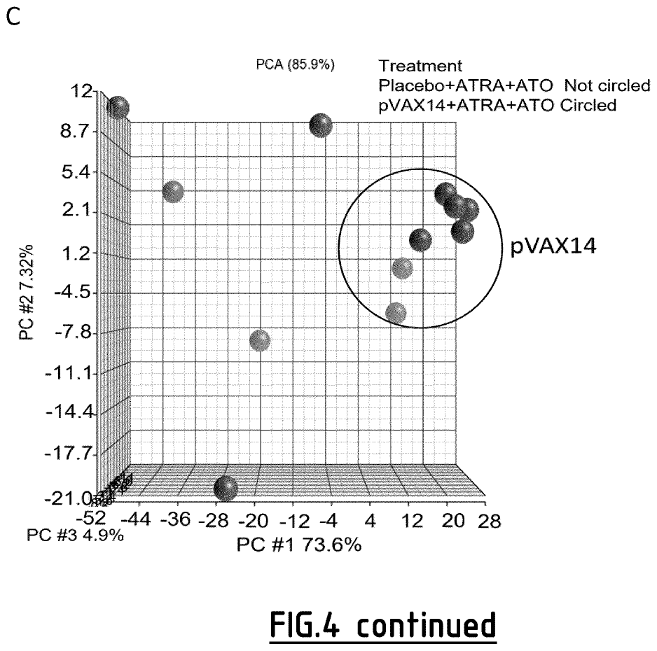

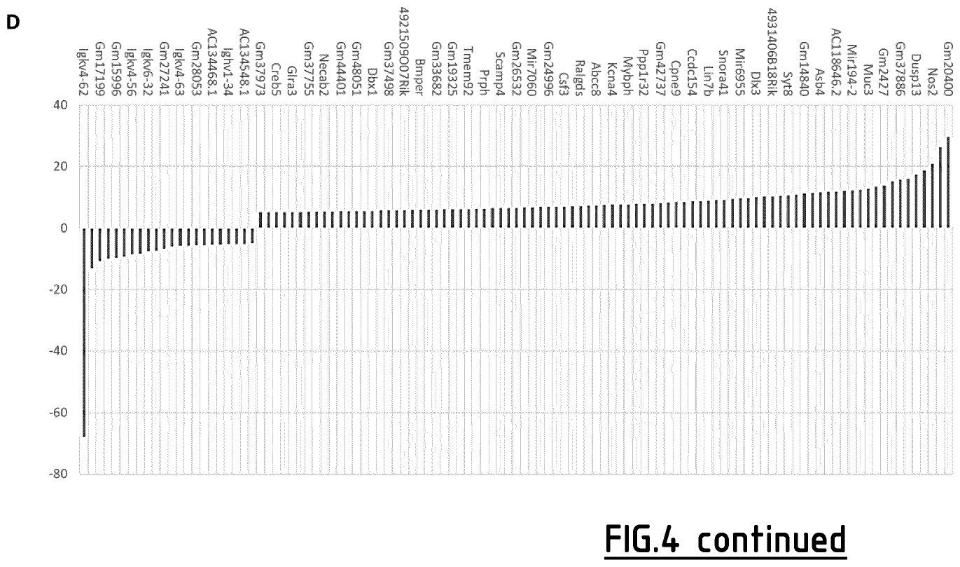

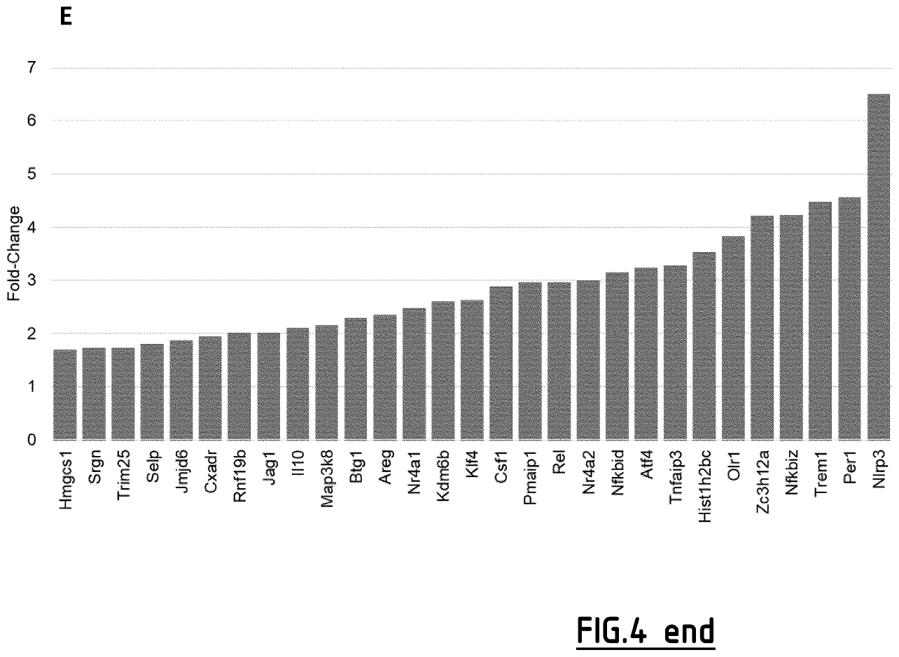

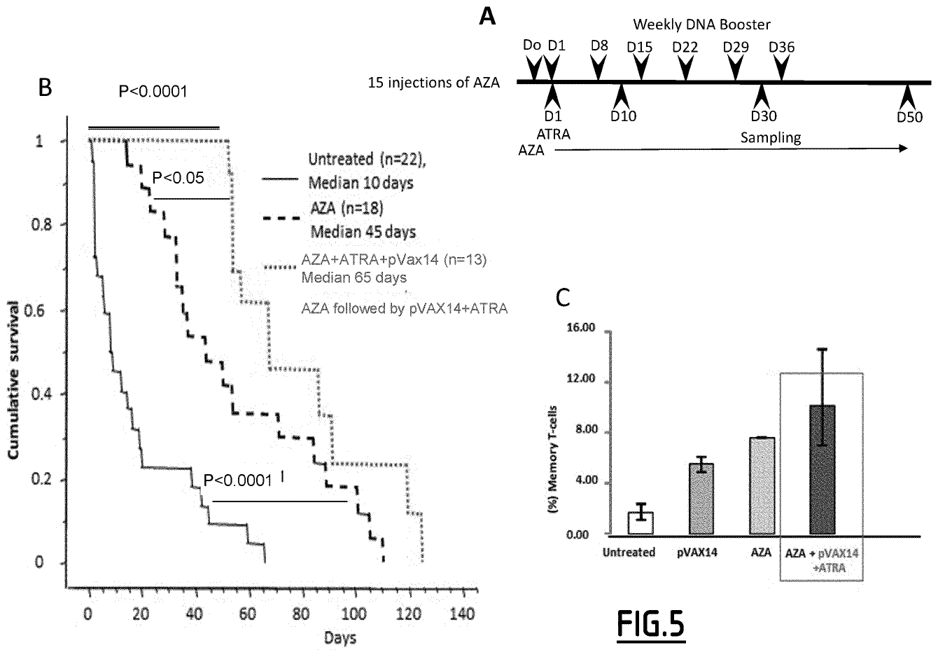

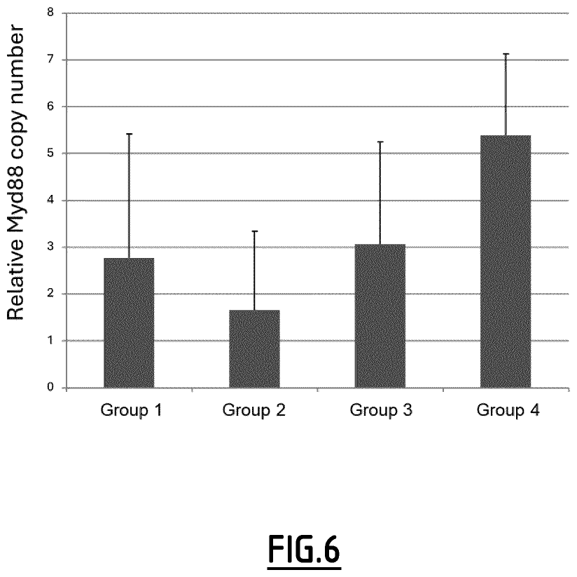

pVax14+ATRA activates the inflammasome pathway with increased expression of Nlrp3 when effectors from immunized mice are co-cultured with leukemia bone marrow (BM), compared with normal FVB/N BM; the increase in apoptosis can be measured using labelled annexin V and the Incucyte and Caspase 1 detection by western blot analyses (see FIGS. 3A, B and C).

pVAX14 added to ATRA+ATO changes the gene expression pattern of mice compared with mice without pVAX14 treatment, with a more homogeneous profile (FIG. 4C). Waterfall plot showing some of the genes regulated are also shown in FIG. 4D. Pathway analyses were undertaken using the DAVID software using the Anova with p=0.01 gene list to generate the enrichment using the top 100 genes (data not shown), which allowed identifying 16 immune related pathways (see Table 1) and a waterfall plot identify 29 biomarkers of response (see FIG. 4E).

TABLE 1
Genes regulated in 16 Immune related pathways

| | Observed | False | |
|---|---|---|---|
| Pathway | gene | discovery | matching proteins in your |
| description | count | rate | network (IDs) |
| Defense response | 16 | 0.000246 | Csf1, Cxadr, Hist1h2bc, Il10, Kdm6b, |
| | | | Nfkbid, Nfkbiz, Nlrp3, Olr1, Pmaip1, Rel, |
| | | | Rnf19b, Selp, Tnfaip3, Trem1, Trim25 |
| Inflammatory | 10 | 0.00112 | Csf1, Il10, Kdm6b, Nfkbid, Nfkbiz, Nlrp3, |
| response | | | Olr1, Rel, Selp, Tnfaip3 |
| Negative regulation of | 5 | 0.00199 | Klf4, Nfkbid, Nlrp3, Tnfaip3, Zc3h12a |
| NF-kappaB | | | |
| transcription factor | | | |
| activity | | | |
| Immune system | 18 | 0.00212 | Csf1, Cxadr, Fst, Hist1h2bc, Il10, Jag1, |
| process | | | Jmjd6, Kdm6b, Klf4, Map3k8, Nlrp3, Olr1, |
| | | | Rel, Rnf19b, Selp, Tnfaip3, Trem1, Trim25 |
| Immune effector | 8 | 0.00702 | Cxadr, Jag1, Nlrp3, Pmaip1, Rnf19b, |
| process | | | Tnfaip3, Trem1, Trim25 |
| Negative regulation of | 4 | 0.00702 | Nfkbid, Nlrp3, Per1, Tnfaip3 |
| I-kappaB kinase/NF- | | | |
| kappaB signaling | | | |
| Negative regulation of | 6 | 0.00979 | Klf4, Nlrp3, Rel, Srgn, Tnfaip3, Zc3h12a |
| cytokine production | | | |
| Immune response | 11 | 0.0124 | Csf1, Hist1h2bc, Il10, Jag1, Kdm6b, Nlrp3, |
| | | | Rel, Rnf19b, Tnfaip3, Trem1, Trim25 |
| Negative regulation of | 2 | 0.0221 | Il10, Tnfaip3 |
| chronic inflammatory | | | |
| response | | | |
| Cytokine secretion | 3 | 0.0221 | Nlrp3, Rnf19b, Trem1 |
| Defense response to | 7 | 0.0286 | Cxadr, Hist1h2bc, Il10, Nlrp3, Pmaip1, |
| other organism | | | Trem1, Trim25 |
| Response to peptide | 7 | 0.0286 | Areg, Btg1, Il10, Jag1, Klf4, Tnfaip3, |
| | | | Uprt |
| Negative regulation of | 3 | 0.0316 | Il10, Tnfaip3, Zc3h12a |
| interleukin-6 | | | |
| production | | | |
| Regulation of cytokine | 8 | 0.0375 | Atf4, Klf4, Nlrp3, Per1, Rel, Srgn, |
| production | | | Tnfaip3, Zc3h12a |
| Negative regulation of | 3 | 0.0383 | Il10, Nlrp3, Srgn |
| cytokine secretion | | | |
| Cellular response to | 7 | 0.0383 | Hmgcs1, Klf4, Nlrp3, Nr4a1, Nr4a2, |
| lipid | | | Tnfaip3, Zc3h12a |

The gene list identified Nlrp3 and Tnfaip3 as most frequently regulated genes (see Table 2).

TABLE 2
Analysis of immune related pathways showing gene list (regulated in
pathways) from 16 immune related pathways. The immunological cell
death gene (Nlrp3) and tumour necrosis factor alpha (Tnfaip3) are
the most frequently regulated genes. The list comprises 29 genes
upregulated (with the number of pathways indicated) (from Table 1).

| | | |
|---|---|---|
| Nlrp3—immunological cell | Cxadr—CXADR Ig-Like Cell | Per1—Period Circadian |
| death (13) | Adhesion Molecule (4) | Regulator 1 (2) |
| Tnfaip3—Tumour necrosis | Hist1h2bc—H2B Clustered | Areg—epidermal growth |
| factor alpha-induced protein | Histone 4 (4) | factor (EGF) family (1) |
| 3 (13) | | |
| Il10—Interleukin 10 cytokine | Jag1—the ligand for the | Atf4—activating |
| (9) | receptor Notch 1 (4) | transcription factor 4 (1) |
| Klf4—Kruppel-like factor 4 | Kdm6b—Lysine | Btg1 encodes an anti- |
| (6) | Demethylase 6B (4) | proliferation factor 1 (1) |
| Rel—Proto-Oncogene, NF- | Nfkbid—NFKB Inhibitor | Hmgcs1—3-Hydroxy-3- |
| KB Subunit (6) | Delta (4) | Methylglutaryl-CoA |
| | | Synthase 1 (1) |
| Trem1—Triggering receptor | Olr1—Oxidized low-density | Jmjd6—Jumonji Domain |
| expressed on myeloid | lipoprotein receptor 1 (3) | Containing 6 (1) |
| cells 1 (6) | | |
| Rnf19b—Ring Finger | Pmaip1—Phorbol-12- | Map3k8—Mitogen- |
| Protein 19B (5) | Myristate-13-Acetate- | Activated Protein Kinase |
| | Induced Protein 1 (3) | Kinase Kinase 8 (1) |
| Trim25—Tripartite Motif | Selp—Selectin P (3) | Nr4a1—Nuclear Receptor |
| Containing 25 (5) | | Subfamily 4 Group A |
| | | Member 1 (1) |
| Zc3h12a—Zinc Finger | Srgn—Serglycin (3) | Nr4a2—Nuclear Receptor |
| CCCH-Type Containing | | Subfamily 4 Group A |
| 12A (5) | | member 2 (1) |
| CSF1—Fms, - colony | Nfkbiz—(NF-kappa-B | |
| stimulating Factor (4) | inhibitor zeta (2) | |

The addition of pVAX14 and ATRA following azacytidine prolongs survival and increases memory T-cells compared to azacytidine alone and untreated HR-MDS mice (FIG. 5).

### REFERENCES

1. Furugaki K, Pokorna K, Le Pogam C, Aoki M, Reboul M, Bajzik V, Krief P, Janin A, Noguera ME, West R, Charron D, Chomienne C, Pla M, Moins-Teisserenc H, Padua R A. DNA vaccination with all-trans retinoic acid treatment induces long-term survival and elicits specific immune responses requiring CD4+ and CD8+ T-cell activation in an acute promyelocytic leukemia mouse model. Blood 15:653-6, 2010. Epub 2009 Nov. 19. PMID:19965687
2. Gorombei P, Guidez zF, Ganesan S, Chiquet M, Pellagatti A, Goursaud L, Tekin N, Beurlet S, Patel S, Guerenne L, Le Pogam C, Setterblad N, de al Grange P, Leboeuf C, Janin A, Noguera M-E, Sarda-Mantel L, Merlet P, Boultwood J, Konopleva M, Andreef M, West R, Pla M, Ades L, Fenaux P, Krief P, Chomiemme C, Omidvar N, Padua R A, BCL-2 inhibitor ABT-737 effectively targets leukemia-initiating cells with differential regulation of relevant genes leading to extended survival ina NRAS/BCL-2 mouse model of high risk-myelodysplastic syndrome. Int J Mol Sci 22:10658. 2021. PMID: 34638998
3. Le Pogam C, Patel S, Gorombei P, Guerenne L, Krief P, Bernasconi E, Tekin N, Sicre F, Schlageter M-E, Chopin M, Mathews V, West RR, PLA M, Fenaux P, Chomienne C, Padua R A. DNA-mediated immunotherapy induces immune responses and extends life-span in two different mouse models of myeloid malignancies. Oncotarget, 6:32494-508, 2015 PMID:26378812
4. Omidvar N, Kogan S, Beurlet S, Le Pogam C, Janin A, West R, Noguera M E, Reboul M., Soulie A., Le Bouf C, Setterblad N, Felsher D, Lagasse, E, Mohamedali A, Thomas NS, Fenaux P, Fountenay M, Pla M, Mufti G J, Weissmann I, Chomienne C, Padua R A. BCL-2 and mutant NRAS interact physically and functionally in a mouse model of progressive myelodysplasia. Cancer Research 67:11657-67, 2007. PMID:18089795
5. Padua R A, Larghero J, Robin M, le Pogam C, Schlageter M-H, Muszlak S, Fric J, West R, Rousselot P, Phan T H, Mudder L, Triesserenc H, Carpentier A, Kogan S, Degos L, Pla M, Bishop M, Stevenson F K, Charron D, Chomienne C. PML-RARA-targeted DNA vaccine induces protective immunity in a mouse model of leukemia. Nature Medicine 9:1413-1417, 2003. PMID: 14566333
6. Patel S, Guerenne L, Gorombei P, Omidvar N, Schlageter M, Alex A A, Ganesan S, West R, Ades L, Mathews V, Krief P, Pla M, Fenaux P, Chomienne C, Padua R A. PVAX14-mediated add-on immunotherapy combined with arsenic trioxide and all-trans retinoic acid (ATO+ATRA) targeted therapy effectively increases survival of APL mice. Blood Cancer Journal, Dec 11;5:e374. doi: 10.1038/bcj.2015.102 PMID:26657197
7. Pokorna K, Chopin M, Le Pogam C, Balitrand N, Reboul M, Cassinat B, Chomienne C, Padua R A, Pla M. Tracking of oncogene positive cells in preclinical models: biomarker of long term drug efficacy. Molecular and Cellular Probes 27:1-5, 2013. PMID: 22906630
8. Robin M, Andreu-Gallien J, Schlageter M-H, Bengoufa D, Guillemot I, Pokorna K, Robert C, Larghero J, Rousselot P, Raffoux E, Dombret H, Fenaux P, Pla M, Charron D, Padua R A, Chomienne C. Frequent antibody production against RARA in both APL mice And patients. Blood 108:1972-1974, 2006. PMID: 16728697
9. Teixeira L, Medioni J, Garibal J, Adotevi O, Doucet L, Durey M-A D, Ghrieb Z, Kiladjian J-J, Brizard M, Laheurte C, Wehbe M, Pliquet E, Escande M, Defrance R, Cuine S, Oudard S, Wain-Hobson S, Doppler V, Huet, A first-in-human Phase I study of INVAC=1, an optimized human telomerase DNA vaccine in patients with advanced solid tumors. Clinical Cancer Research 2020 Feb. 1;26(3):588-597. doi: 10.1158/1078-0432.CCR-19-1614. Epub 2019 Sep. 26.

1. A pharmaceutical combination comprising:
(i) at least one nucleic acid comprising an antisense and flipped sequence of a fragment of the sequence coding for a kanamycin resistance protein, wherein said nucleic acid is capable of eliciting an immune response in a mammal and wherein said nucleic acid is formulated with a nano-vehicle, and
(ii) at least one non-immunosuppressive inducer of tumor cell apoptosis.

2. The pharmaceutical combination according to claim 1, wherein said nucleic acid comprises a sequence selected from the group consisting of:
a) sequence SEQ ID NO: 2,
b) a sequence at least 80% identical to SEQ ID NO: 2,
c) a fragment of at least 50 consecutive nucleotides of SEQ ID NO: 2, and
d) sequence SEQ ID NO: 3.

3. The pharmaceutical combination according to claim 1, wherein said antisense and flipped sequence encodes at least one immunogenic peptide selected from the group consisting of sequence SEQ ID NO: 4 and SEQ ID NO: 5.

4. The pharmaceutical combination according to claim 1, wherein said non-immunosuppressive inducer of tumor cell apoptosis is ATRA, arsenic, arsenic trioxide or azacytidine.

5. The pharmaceutical combination according to claim 1, wherein said nano-vehicle is a tetra-functional amphiphilic block copolymer of formula (I): 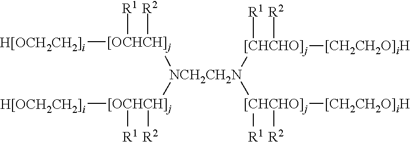
or an organic or mineral salt thereof,
in which i and j represent, independently of one another, an integer of between 1 and 500, and
for each R1, R2 pair, one is hydrogen and the other is a methyl group.

6. The pharmaceutical combination according to claim 1, wherein said nucleic acid (i) and said non-immunosuppressive inducer of tumor cell apoptosis (ii) are each provided in the form of one or more doses of a pharmaceutical composition.

7. A method of treatment comprising administering to a subject in need thereof the pharmaceutical combination according to claim 1.

8. The method of claim 7, in the treatment of cancer or an infectious disease.

9. The method according to claim 7, wherein said nucleic acid (i) and said non-immunosuppressive inducer of tumor cell apoptosis (ii) are administered simultaneously or sequentially.

10. The method according to claim 7, wherein said nucleic acid (i) and/or said non-immunosuppressive inducer of tumor cell apoptosis (ii) is/are administered by intradermal, intra-epidermal, subcutaneous, intramuscular, oral route or intratumoral route.

11. The method according to claim 7, wherein
said nucleic acid (i) is administered at a dosage of from 500 μg to 2 mg, and/or
said non-immunosuppressive inducer of tumor cell apoptosis (ii) is ATRA and is administered at an oral dosage of 45 mg/m².

12. A method for assessing activation of an immune response, wherein said method comprises measuring the quantity of at least 5 biomarkers selected from the group consisting of NLRP3, TNFAIP3, PMAIP1, IL10, KLF4, REL, TREM1, RNF19b, TRIM25, ZCH12A, CSF1-FMS, CXADR, HISTIH2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2 in a biological sample.

13. A method for monitoring response to a treatment based on the pharmaceutical combination according to claim 1 in a subject, wherein said method comprises:
a) measuring the quantity of at least 5 biomarkers selected from the group consisting of NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2 in a biological sample from said subject,
b) comparing the results obtained at step a) with a corresponding control value and/or to a corresponding value from said subject measured before beginning the treatment or measured during the course of treatment,
c) deducing if said subject responds favorably to the treatment, and
d) optionally, repeating steps a) to c).

14. A kit suitable for assessing activation of an immune response, wherein said kit comprises means for detecting at least 5 biomarkers selected from the group consisting of NLRP3, TNFAIP3, PMAIP1, IL10, KLF4, REL, TREM1, RNF19b, TRIM25, ZCH12A, CSF1-FMS, CXADR, HISTIH2BC, JAG1, KDM6B, NFKBID, OLR1, SELP, SRGN, NFKBIZ, PER1, AREG, ATF4, BTG1, HMGCS1, JMJD6, MAP3K8, NR4A1 and NR4A2.

15. The method according to claim 12, wherein said biomarkers comprise NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP and SRGN.

16. An in vitro method for assessing activation of an immune response comprising using a kit according to claim 14.

17. The method according to claim 13, wherein said biomarkers comprise NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP and SRGN.

18. The kit according to claim 14, wherein said biomarkers comprise NLRP3, TNFAIP3, PMAIP1, IL10, KLF4, REL, TREM1, RNF19b, TRIM25, ZCH12A, CSF1-FMS, CXADR, HIST1H2BC, JAG1, KDM6B, NFKBID, OLR1, SELP and SRGN.


SEQUENCE LISTING (7 sequences)

SEQ ID NO: 1 - DNA, 472 bp, synthetic construct
  source 1..472: mol_type = other DNA
  misc_feature 1..472: note = Antisense and flipped sequence of a fragment of the sequence coding for the kanamycin resistance protein
gatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcat

SEQ ID NO: 2 - DNA, 825 bp, synthetic construct
  source 1..825: mol_type = other DNA
  misc_feature 1..825: note = Flipper region
  misc_feature 1..472: note = Antisense and flipped sequence of a fragment of the sequence coding for the kanamycin resistance protein
  misc_feature 473..825: note = Antisense and flipped sequence of a fragment of the pVax1 plasmid
gatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcac

SEQ ID NO: 3 - DNA, 3540 bp, synthetic construct
  source 1..3540: mol_type = other DNA
  misc_feature 1..3540: note = pVax14
  misc_feature 763..1587: note = Flipper region
  misc_feature 763..1234: note = Antisense and flipped sequence of a fragment of the sequence coding for the kanamycin resistance protein
  misc_feature 1235..1587: note = Antisense and flipped sequence of a fragment of pVax1
  misc_feature 1816..2610: note = Sequence coding for the kanamycin resistance protein
tgctggccttttgctcacatgttcttgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttcgcttgctgtccataaaaccgcccagtagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatgaaaaacgccagcaacgcggc

SEQ ID NO: 4 - protein, 35 aa, synthetic construct
  REGION 1..35: note = Immunogenic polypeptide
MRCFAWWSNGQVAGSSVCSRRIASAMMDTFSAGAR

SEQ ID NO: 5 - protein, 35 aa, synthetic construct
  REGION 1..35: note = Immunogenic polypeptide
MFRLVVEWAGSRIKRMQPPHCISHDGYFLGRSKVR

SEQ ID NO: 6 - protein, 59 aa, synthetic construct
  REGION 1..59: note = Immunogenic polypeptide
MPAIVFPILPLAVLPHPTPQNRMTPTQTMRCNFLILLGKDSGSGTFQGQGRHGGGLGRQ

SEQ ID NO: 7 - DNA, 2999 bp, synthetic construct
  misc_feature 1..2999: note = pVAX1 vector
  regulatory 137..724: note = promoter - CMV promoter; regulatory_class = promoter
  regulatory 664..683: note = promoter - T7 promoter/priming site; regulatory_class = promoter
  misc_feature 696..811: note = Multiple cloning site
  misc_feature 1226..2020: note = sequence coding for the kanamycin resistance protein
  rep_origin 2320..2993: note = pUC origin
  source 1..2999: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt